(19) United States
(12) Patent Application Publication
(10) Pub. No.: US 2016/0310383 A1
(43) Pub. Date: Oct. 27, 2016

(54) METHOD FOR DYEING KERATINOUS SUBSTANCES STARTING FROM COLOURED OLIGOMERS AND/OR POLYMERS RESULTING FROM META-PHENYLENEDIAMINES, COMPOSITION AND DYEING AGENT

(71) Applicant: L'OREAL, Paris (FR)

(72) Inventors: Stéphane SABELLE, Paris (FR); Madeleine LEDUC, Paris (FR)

(21) Appl. No.: 15/104,163

(22) PCT Filed: Dec. 10, 2014
(86) PCT No.: PCT/EP2014/077222

§ 371 Date: Jun. 13, 2016

(30) Foreign Application Priority Data
Dec. 13, 2013 (FR) 1362551

(51) Int. Cl. A61K 8/41; A61K 8/84; C07C 217/84; C08G 73/02; C09B 57/00; C07C 233/43; C08G 69/00; A61K 8/368; A61K 8/34; A61K 8/19; A61Q 5/06; C07C 211/50

(52) U.S. Cl. CPC A61K 8/411; A61Q 5/065; A61K 8/84; C07C 217/84; C08G 73/026; C07C 211/50; C07C 233/43; C08G 69/00; A61K 8/368; A61K 8/34; A61K 8/19; C09B 57/00; A61K 2800/4322; A61K 2800/5922; A61K 2800/594

(57) ABSTRACT

A subject-matter of the invention is a) a method for dyeing keratinous substances starting from a dyeing agent chosen from coloured oligomers and polymers obtained from the polycondensation of i) meta-phenylenediamine (MPD) couplers in the presence of ii) one or more ingredients chosen from radical initiators and/or chemical oxidizing agents, b) a composition comprising one or more coloured oligomers and/or one or more coloured polymers as defined above and c) a coloured oligomeric or polymeric dyeing agent obtained by reaction between i) and ii) as defined above, and the use of the coloured oligomer(s) or polymer(s) for dyeing keratinous substances. The dyeing method and the composition according to the invention make it possible in particular to obtain a long-lasting colouration on keratinous fibres which is vivid, chromatic and/or homogeneous.

A subject-matter of the invention is a) a method for dyeing keratinous substances, in particular keratinous fibres, such as the hair, starting from a dyeing agent chosen from coloured oligomers and polymers obtained from the polycondensation of i) meta-phenylenediamine couplers (MPDs) in the presence of ii) one or more ingredients chosen from radical initiators and/or chemical oxidizing agents, b) a composition comprising one or more coloured oligomers and/or one or more coloured polymers as defined above and c) a coloured oligomer or polymer dyeing agent obtained by reaction between i) and ii) as defined above and the use of the coloured oligomer(s) or polymer(s) to dye keratinous fibres.

It is known to dye keratinous fibres by direct dyeing or semi-permanent dyeing. Direct dyeing or semi-permanent dyeing consists in introducing the colour via a coloured molecule which becomes adsorbed at the surface of the individual hair or which penetrates into the individual hair. Thus, the method conventionally used in direct dyeing consists in applying direct dyes to the keratinous fibres, which direct dyes are coloured and dyeing molecules having an affinity for the fibres, in leaving the fibres in contact with the dyeing molecules and in then optionally rinsing the fibres. Generally, this technique results in chromatic colourations.

Currently, the main direct dyes used in hair dyeing are dyes having low molecular weights. Due to their size, they generally exhibit a good affinity for the hair fibre. However, due to their solubility in water and their size, they have low persistences towards shampooing operations. Furthermore, natural and fundamental colours are generally difficult to achieve.

The synthesis of direct dyes having high molecular weights has also been described (WO 2011/006946, WO 2011/113676 and WO 2011/113675). A priori, these coloured polymers do not penetrate into the hair fibre. Instead, they have a tendency to be deposited around the keratinous fibres, which brings about in particular problems of feel and/or of persistences towards shampooing operations.

There thus exists a real need to find novel direct dyes which are capable of connecting securely to the hair fibre, of having excellent persistences, in particular towards shampooing, of exhibiting a very good feel and of achieving natural and fundamental colours and/or which provide a colouration of the keratinous fibres which is not very selective (i.e., for which the difference in colouration along one and the same keratinous fibre between its tip and its root is low, or homogeneous from one fibre to another).

This aim is achieved with the present invention, a first subject-matter of which is a method for dyeing keratinous substances, in particular keratinous fibres, preferably human keratinous fibres, such as the hair, employing at least one dyeing agent obtained from the polycondensation of i) MPD couplers which react with one another in the presence of ii) one or more ingredients chosen from radical initiators and/or chemical oxidizing agents.

Another subject-matter is a dyeing agent obtained from the polycondensation of i) MPD couplers which react with one another in the presence of ii) at least one radical initiator and/or at least one chemical oxidizing agent.

Another subject-matter of the invention is a composition comprising at least one dyeing agent as defined above, preferably not comprising an oxidation base and/or hydrogen peroxide.

Another subject-matter relates to the use of the dyeing agent as defined above for dyeing keratinous substances, in particular keratinous fibres, preferably human keratinous fibres, such as the hair.

The colourations obtained with the dyeing method, with the composition or starting from the dyeing agent(s) of the invention are attractive, vivid, chromatic and/or very persistent with regard to common attacking factors, such as the sun, perspiration or sebum and towards other hair treatments, such as successive shampooing operations, while respecting the keratinous fibres. The vividness obtained is particularly noteworthy. It is the same for the homogeneity of the colour of the keratinous fibres, i.e. of low selectivity from the root to the tip.

Within the meaning of the present invention and unless otherwise indicated:
“Dyeing agent” is understood to mean an oligomer and/or polymer resulting from a linear or non-linear covalent assemblage, with a number of greater than or equal to 2 repeat units or monomer units, resulting from the polycondensation of MPD couplers solely with one another, i.e. the oligomeric and/or polymeric chain does not comprise a monomer unit originating from an oxidation base, for example; the said dyeing agent has a high molecular weight, in particular of greater than or equal to 230 g/mol, preferably of greater than 300 g/mol;
“Oligomer” is understood to mean a compound comprising from 2 to 4 monomer units resulting from the polycondensation of MPD couplers solely with one another, i.e. devoid, in the oligomeric chain, of a monomer unit resulting from an oxidation base;
“Polymer” is understood to mean a compound comprising at least 5 monomer units and preferably between 5 and 100 monomer units resulting from the polycondensation of MPD couplers solely with one another, i.e. devoid, in the polymeric chain, of a monomer unit resulting from an oxidation base;
the “aryl” or “heteroaryl” radicals or the aryl or heteroaryl part of a radical can be substituted by at least one substituent carried by a carbon atom chosen from:
   a C₁-C₈ alkyl radical optionally substituted by one or more radicals chosen from the following radicals: hydroxyl, C₁-C₄ alkoxy, C₂-C₄ (poly)hydroxyalkoxy, acylamino or amino substituted by two identical or different C₁-C₄ alkyl radicals which optionally carry at least one hydroxyl group or it being possible for the two radicals to form, with the nitrogen atom to which they are attached, a saturated or unsaturated and optionally substituted 5- to 7-membered and preferably 5- or 6-membered heterocycle optionally comprising another heteroatom identical to or different from nitrogen;
   a halogen atom;
   a hydroxyl group;
   a C₁-C₄ alkoxy radical;
   a C₂-C₄ (poly)hydroxyalkoxy radical;
   an amino radical;
   an amino radical substituted by one or two identical or different C₁-C₆ alkyl radicals optionally carrying at least:
      i) one hydroxyl group,
      ii) an amino group optionally substituted by one or two C₁-C₃ alkyl radicals which are optionally substituted,
      iii) a quaternary ammonium group —N⁺R′R″R′″ M⁻ for which R′, R″ and R′″, which are identical or different, represent a hydrogen atom or a C₁-C₄ alkyl group and M represents the anionic counterion,
   an acylamino (—NR—C(O)—R′) radical in which the R radical is a hydrogen atom or a C₁-C₄ alkyl radical optionally carrying at least one hydroxyl group and the R′ radical is a C₁-C₄ alkyl radical;
   a carbamoyl ((R)₂N—C(O)—) radical in which the R radicals, which are identical or different, represent a hydrogen atom or a C₁-C₄ alkyl radical optionally carrying at least one hydroxyl group;
   an alkylsulfonylamino (R′—S(O)₂—N(R)—) radical in which the R radical represents a hydrogen atom or a C₁-C₄ alkyl radical optionally carrying at least one hydroxyl group and the R′ radical represents a C₁-C₄ alkyl radical, a phenyl radical;
   an aminosulfonyl ((R)₂N—S(O)₂—) radical in which the R radicals, which are identical or different, represent a hydrogen atom or a C₁-C₄ alkyl radical optionally carrying at least one hydroxyl group;
   a carboxyl radical in the acid or salified form (preferably in the form salified with an alkali metal or a substituted or unsubstituted ammonium);
   a cyano group;
   a nitro or nitroso group;
   a polyhaloalkyl group, preferably trifluoromethyl;
the cyclic or heterocyclic part of a non-aromatic radical can be substituted by at least one substituent chosen from the following groups:
   hydroxyl;
   C₁-C₄ alkoxy or C₂-C₄ (poly)hydroxyalkoxy;
   C₁-C₄ alkyl;
   alkylcarbonylamino (R—C(O)—NR′—), in which the R′ radical is a hydrogen atom or a C₁-C₄ alkyl radical optionally carrying at least one hydroxyl group and the R radical is a C₁-C₂ alkyl radical or an amino radical optionally substituted by one or two identical or different C₁-C₄ alkyl groups themselves optionally carrying at least one hydroxyl group;
   alkylcarbonyloxy (R—C(O)—O—), in which the R radical is a C₁-C₄ alkyl radical or an amino group optionally substituted by one or two identical or different C₁-C₄ alkyl groups themselves optionally carrying at least one hydroxyl group;
   alkoxycarbonyl or aminocarbonyl (R-G-C(O)—), in which the R radical is a C₁-C₄ alkyl radical and G is an oxygen atom or an amino group optionally substituted by a C₁-C₄ alkyl group itself optionally carrying at least one hydroxyl group;
a cyclic or heterocyclic radical or a non-aromatic part of an aryl or heteroaryl radical can also be substituted by one or more oxo groups;
an “aryl” radical represents a monocyclic or fused or non-fused polycyclic carbon-based group comprising from 6 to 22 carbon atoms, at least one ring of which is aromatic; preferably, the aryl radical is a phenyl, biphenyl, naphthyl, indenyl, anthracenyl or tetrahydronaphthyl and more preferably phenyl;
a “heteroaryl radical” represents an optionally cationic, 5- to 22-membered, monocyclic or fused or non-fused polycyclic group comprising from 1 to 6 heteroatoms chosen from nitrogen, oxygen, sulfur and selenium atoms (preferably chosen from N, O and S), at least one ring of which is aromatic; preferably, a heteroaryl radical is chosen from acridinyl, benzimidazolyl, benzobistriazolyl, benzopyrazolyl, benzopyridazinyl, benzoquinolyl, benzothiazolyl, benzotriazolyl, benzoxazolyl, pyridinyl, tetrazolyl, dihydrothiazolyl, imidazopyridyl, imidazolyl, indolyl, isoquinolyl, naphthoimidazolyl, naphthoxazolyl, naphthopyrazolyl, oxadiazolyl, oxazolyl, oxazolopyridyl, phenazinyl, phenoxazolyl, pyrazinyl, pyrazolyl, pyrilyl, pyrazoyltriazyl, pyridyl, pyridinoimidazolyl, pyrrolyl, quinolyl, tetrazolyl, thiadiazolyl, thiazolyl, thiazolopyridinyl, thiazoylimidazolyl, thiopyrylyl, triazolyl, xanthyl and its ammonium salt;
a “cationic heteroaryl radical” is a heteroaryl group as defined above which comprises an endocyclic or exocyclic quaternized cationic group,
   when the cationic charge is endocyclic, it is included in the electron delocalization via the mesomeric effect; for example, it is a pyridinium, imidazolium or indolinium group:

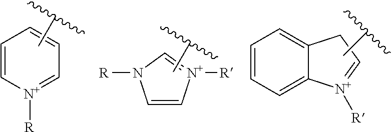

with R and R′ being a heteroaryl substituent as defined above and in particular a (hydroxy)(C₁-C₈)alkyl group, such as methyl; such as N—(C₁-C₄)alkylimidazolium;
when the cationic charge is exocyclic, it concerns, for example, an ammonium or phosphonium R⁺ substituent, such as trimethylammonium, which is outside the heteroaryl, such as pyridinyl, indolyl, imidazolyl or naphthalimidyl, in question:

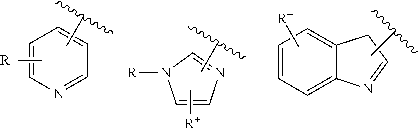

with R a heteroaryl substituent as defined above and R⁺ an ammonium RₐR_{b}R_{c}N⁺−, phosphonium RₐR_{b}R_{c}P⁺— or ammonium RₐR_{b}R_{c}N⁺−(C₁-C₆)alkylamino group with Rₐ, R_{b} and R_{c}, which are identical or different, representing a hydrogen atom or a (C₁-C₈)alkyl group, such as methyl;
a “heterocyclic radical” is a 5- to 22-membered monocyclic or fused or non-fused polycyclic radical which can comprise one or two unsaturations but is non-aromatic, comprising from 1 to 6 heteroatoms chosen from nitrogen, oxygen, sulfur and selenium (preferably chosen from N, O and S);
a “cationic heterocyclic radical” is a saturated, mono, 5- to 7-membered radical as defined above which comprises from 1 to 3 heteroatoms chosen from nitrogen, oxygen and sulfur atoms (preferably chosen from N and O) and which additionally comprises a cationic charge carried by a nitrogen atom present in the heterocyclic ring, such as N—(C₁-C₄)alkylpyrrolidinium, N—(C₁-C₄)alkylpiperidinium, N,N′-di(C₁-C₄)alkylpiperazinium or N—(C₁-C₄)alkylmorpholinium,
a “heterocycloalkyl radical” is a heterocyclic radical comprising at least one saturated ring;
an “alkyl radical” is a linear or branched C₁-C₂₀ and preferably C₁-C₈ hydrocarbon radical;
the expression “optionally substituted” assigned to the alkyl radical is understood to mean that the said alkyl radical can be substituted by one or more radicals chosen from the following radicals: i) hydroxyl, ii) C₁-C₆ alkoxy, iii) acylamino, iv) amino optionally substituted by one or two identical or different C₁-C₄ alkyl radicals, it being possible for the said alkyl radicals to form, with the nitrogen atom which carries them, a 5- to 7-membered heterocycle optionally comprising another nitrogen or non-nitrogen heteroatom; v) or a quaternary ammonium group —N⁺R′R″R′″ M⁻ for which R′, R″ and R′″, which can be identical or different, represent a hydrogen atom or a C₁-C₄ alkyl group, or alternatively —N⁺R′R″R′″ forms a heteroaryl, such as imidazolium optionally substituted with a C₁-C₄ alkyl group, and M⁻ represents the anionic counterion;
an “alkoxy radical” is an alkyl-oxy radical for which the alkyl radical is a linear or branched C₁-C₁₀ and preferably C₁-C₈ hydrocarbon radical;
an “alkylthio radical” is an alkyl-S— radical for which the alkyl radical is a linear or branched C₁-C₁₀ and preferably C₁-C₈ hydrocarbon radical;
when the alkoxy or alkylthio group is optionally substituted, this is understood to mean that the alkyl group is optionally substituted as defined above;
“organic or inorganic acid salt” is understood more particularly to mean the salts chosen from a salt derived from i) hydrochloric acid HCl, ii) hydrobromic acid HBr, iii) sulfuric acid H₂SO₄, iv) alkylsulfonic acids: Alk-S(O)₂OH, such as methanesulfonic acid and ethanesulfonic acid; v) arylsulfonic acids: Ar—S(O)₂OH, such as benzenesulfonic acid and toluenesulfonic acid; vi) citric acid; vii) succinic acid; viii) tartaric acid; ix) lactic acid; x) alkoxysulfinic acids: Alk-O—S(O)OH, such as methoxysulfinic acid and ethoxysulfinic acid; xi) aryloxysulfinic acids, such as tolueneoxysulfinic acid and phenoxysulfinic acid; xii) phosphoric acid H₃PO₄; xiii) acetic acid CH₃C(O)—OH; xiv) triflic acid CF₃SO₃H; and xv) tetrafluoroboric acid HBF₄;
“anionic counterion” is understood to mean an anion or an anionic group resulting from an organic or inorganic acid salt which counterbalances the cationic charge of the dye; more particularly, the anionic counterion is chosen from: i) halides, such as chloride or bromide; ii) nitrates; iii) sulfonates, including C₁-C₆ alkanesulfonates: Alk-S(O)₂O⁻, such as methanesulfonate or mesylate and ethanesulfonate; iv) arenesulfonates: Ar—S(O)₂O⁻, such as benzenesulfonate and toluenesulfonate or tosylate; v) citrate; vi) succinate; vii) tartrate; viii) lactate; ix) alkylsulfates: Alk-O—S(O)O⁻, such as methylsulfate and ethylsulfate; x) arylsulfates: Ar—O—S(O)O⁻, such as benzenesulfate and toluenesulfate; xi) alkoxysulfates: Alk-O—S(O)₂O⁻, such as methoxysulfate and ethoxysulfate; xii) aryloxysulfates: Ar—O—S(O)₂O⁻; xiii) phosphates O═P(OH)₂—O⁻, O═P(O⁻)₂—OH, O═P(O⁻)₃, HO—[P(O)(O⁻)]_{w}—P(O)(O⁻)₂ with w being an integer; xiv) acetate; xv) triflate; xvi) borates, such as tetrafluoroborate; and xvii) disulfate (O=)₂S(O⁻)₂ or SO₄²⁻ and monosulfate HSO₄⁻;
the anionic counterion, resulting from organic or inorganic acid salt, ensures the electrical neutrality of the molecule; thus, it is understood that, when the anion comprises several anionic charges, then the same anion can serve for the electrical neutrality of several cationic groups in the same molecule or else can serve for the electrical neutrality of several molecules; for example, a disulfide dye of formula (I) which comprises two cationic chromophores can comprise either two “singly charged” anionic counterions or one “doubly charged” anionic counterion, such as (O=)₂S(O⁻)₂ or O═P(O⁻)₂—OH;
moreover, the addition salts which can be used in the context of the invention are chosen in particular from addition salts with a cosmetically acceptable base, such as alkaline agents as defined below, for example alkali metal hydroxides, such as sodium hydroxide or potassium hydroxide, aqueous ammonia, amines or alkanolamines;
the expression “at least one” is equivalent to the term “one or more”; and
the expression “inclusive” for a range of concentrations means that the limits of the range form part of the interval defined.

### i) Meta-Phenylenediamine or MPD Couplers

The process for the preparation of the dyeing agent(s) of the invention employs one or more identical or different MPD couplers, preferably ones which are identical to one another.

meta-Phenylenediamine or MPD coupler is understood to mean a colourless or weakly coloured compound comprising a benzene group substituted in the 1 and 3 positions by two amino groups which are themselves optionally substituted; the term thus means meta-phenylenediamine (or 3-aminoaniline or 1,3-diaminobenzene) or its derivatives substituted on the nitrogen atoms or on the benzene ring, it not being possible for these, as couplers, to colour in the presence of atmospheric oxygen, and these being known by a person skilled in the art as being able to couple or condense with oxidation bases to generate the colour of the keratinous fibres in the conventional oxidation dyeing method (see, for example, Kirk-Othmer Encyclopedia of Chemical Technology, “Hair Preparation”, 4th Ed., Vol. 12, 1994, p. 904; Ullmann's Encyclopedia of Industrial Chemistry, “Hair Preparation”, 2002, DOI: 10.1002/14356007.a12_571).

More particularly, the MPD couplers of the invention are chosen from those of formula (I) below:

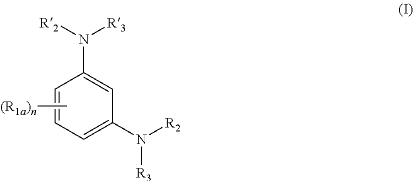

and also their salts of inorganic or organic acids or bases, their optical or geometrical isomers, their tautomers and/or their solvates, such as the hydrates; in which formula (I):
R₁ₐ represents a halogen atom or a group chosen from 1) (C₁-C₈)alkyl optionally substituted by one or more halogen atoms, such as fluorine, or hydroxyl, (di)(C₁-C₆)(alkyl)amino, —N(R)—C(O)—NR′₂, (C₁-C₆)alkoxy or (C₁-C₆)alkylthio groups, 2) (C₁-C₈)alkoxy optionally substituted by one or more halogen atoms or groups chosen from hydroxyl, (di)(C₁-C₄)(alkyl)amino, carboxyl, R—C(O)—N(R′)—, R—S(O)₂—N(R′)—, RR′N—C(O)—, pyrrolidino, piperidino, N′—(C₁-C₄)(alkyl)-N-piperazino, morpholino, N—(C₁-C₄)alkylpyrrolidinium, N—(C₁-C₄)alkylpiperidinium, N,N′-di(C₁-C₄)alkylpiperazinium, N—(C₁-C₄)alkylmorpholinium, N—(C₁-C₄)alkylimidazolium and tri(C₁-C₄)alkylammonium with R and R′, which are identical or different, representing a hydrogen atom or a (C₁-C₄)alkyl group, it being understood that the alkyl group of the alkoxy can be interrupted by one or more heteroatoms, such as oxygen; 3) (C₁-C₈)alkylthio optionally substituted by one or more hydroxyl or R—C(O)—N(R′)— groups with R and R′ as defined above; 4) R—C(O)—N(R′)— with R and R′ as defined above; 5) (C₁-C₄)alkoxycarbonyl; 6) carboxyl; 7) —SO₃H; 8) RR′N—C(O)— with R and R′ as defined above; 9) (di)(C₁-C₄)(alkyl)amino; and 10) R—O—C(O)— with R as defined above;
R₂, R₃, R′₂ and R′₃, which are identical or different, represent i) a hydrogen atom, ii) a (C₁-C₈)alkyl group optionally substituted by one or more of the following groups: a) hydroxyl, b) R′₂—(W)ₚ—C(W)—(W″)_{q}—, such as aminocarbonylamino or guanidino with p and q=0 or 1, R′₂ representing a hydrogen atom or a group chosen from (C₁-C₆)alkyl, W representing an oxygen or sulfur atom or an amino group N(R″), preferably an oxygen atom, and W′ and W″ representing an oxygen or sulfur atom or an N(R′″) group with R″ and R″, which are identical or different, representing a hydrogen atom or a (C₁-C₄)alkyl group, preferably a hydrogen atom, c) (di)(C₁-C₆)(alkyl)amino or d) (C₁-C₈)alkylthio, or iii) an R′₂—(W)ₚ—C(W)—(W″)_{q}— group as defined above;
or else R₂ with R₃ and/or R′₂ with R′₃ form, with the nitrogen atom which carries them, a 5- to 7-membered monocyclic heterocycle, such as pyrrolidino optionally; piperazino, which can be substituted by one or more (C₁-C₆)alkyl groups; (C₁-C₆)(alkyl)piperidino and (di)azepano;
preferably, R′₂ and R′₃ represent a hydrogen atom and R₂ represents a hydrogen atom and R₃ is as defined above, and more particularly R₂ and R₃ represent a hydrogen atom;
n has the value 0, 1, 2 or 3, preferably 1 or 2;
   It being understood that, when the compound of formula (I) comprises one or more cationic substituents, then the compound comprises as many anionic counterions as cationic charges in order to achieve the electrical neutrality of the compound of formula (I).

According to a specific embodiment of the invention, the MPD coupler(s) are chosen from the couplers of following formula (Ia):

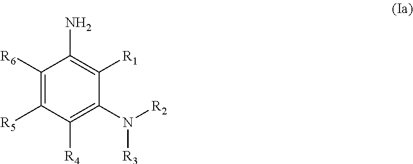

and also their salts of inorganic or organic acids or bases, their optical isomers or geometrical isomers, their tautomers and/or their solvates, such as the hydrates; in which formula (Ia):
R₂ and R₃, which are identical or different, represent i) a hydrogen atom; ii) a (C₁-C₆)alkyl group optionally substituted by one or more groups chosen from: a) hydroxyl, b) urea —N(H)—C(O)—NH₂, c) guanidino —N(H)—C(NH)—NH₂, d) (di)(C₁-C₄)(alkyl)amino or e) (C₁-C₄)alkylthio; iii) a (C₁-C₄)alkylcarbonyl group, such as acetyl CH₃—C(O)—, or iv) an aminocarbonyle H₂N—C(O)— group;
   or else R₂ and R₃ form, together with the nitrogen atom which carries them, a saturated heterocycle, such as pyrrolidino, piperidino or morpholino;
   preferably, R₂ represents a hydrogen atom and R₃ is as defined above, and more particularly R₂ and R₃ represent a hydrogen atom;
R₁, R₄, R₅ and R₆, which are identical or different, represent i) a hydrogen or halogen atom, ii) a (C₁-C₆)alkyl radical optionally substituted by one or more atoms or groups chosen from: a) halogen, b) hydroxyl, c) (di)(C₁-C₆)(alkyl)amino or d) (C₁-C₆)alkylthio, iii) (C₁-C₆)alkoxy optionally substituted by one or more halogen atoms or groups chosen from hydroxyl, (di)(C₁-C₄)(alkyl)amino, carboxyl, (C₁-C₆)alkylcarbonyl, R—C(O)—N(R′)—, R—S(O)₂—N(R′)—, RR′N—C(O)—, cationic heterocycle or cationic heteroaryl, such as N—(C₁-C₄)alkylpyrrolidinium, N—(C₁-C₄)alkylpiperidinium, N,N′-di(C₁-C₄)alkylpiperazinium, N—(C₁-C₄)alkylmorpholinium or N—(C₁-C₄)alkylimidazolium, and tri(C₁-C₄)alkylammonium with R and R′, which are identical or different, representing a hydrogen atom or a (C₁-C₄)alkyl group, it being understood that the alkyl group of the alkoxy iii) can be interrupted by one or more oxygen atoms; iv) (C₁-C₆)alkylthio optionally substituted by one or more groups chosen from hydroxyl, (di)(C₁-C₄)(alkyl)amino and R—C(O)—N(R′)—, such as acetamido, with R and R′ as defined above; v) R—C(O)—N(R′)—, such as acetamido, with R and R′ as defined above; vi) R—O—C(O)— with R as defined above; vii) carboxyl; viii) SO₃H; or ix) aminocarbonyl;
   It being understood that, when the compound of formula (Ia) comprises one or more cationic substituents, then it comprises as many anionic counterions as cationic charges in order to achieve the electrical neutrality of the compound of formula (Ia).

According to an advantageous alternative form of the invention, the MPDs are such that, in the formula (Ia):
R₁ represents a hydrogen atom or a group chosen from (C₁-C₆)alkyl and (C₁-C₆)alkoxy optionally substituted by a hydroxyl group; preferably, R₁ represents a hydrogen atom;
R₂ and R₃, which are identical or different, represent i) a hydrogen atom; ii) a (C₁-C₆)alkyl group optionally substituted by a group chosen from hydroxyl and guanidino; or iii) a (C₁-C₄)alkylcarbonyl group, such as acetyl CH₃—C(O)—;
   or else R₂ and R₃ form, together with the nitrogen atom which carries them, a saturated heterocycle, such as pyrrolidino, piperidino or morpholino; preferably, R₂ represents a hydrogen atom and R₃ is as defined above and more particularly R₂ and R₃ represent a hydrogen atom;
R₄ represents a hydrogen atom;
R₅ represents a hydrogen atom or a group chosen from (C₁-C₆)alkyl optionally substituted by a group which is hydroxy or (di)(C₁-C₄)(alkyl)amino, and (C₁-C₆)alkoxy; preferably, R₅ represents a hydrogen atom;
R₆ represents a hydrogen atom or a group chosen from:
   (C₁-C₆)alkyl, and
   (C₁-C₆)alkoxy optionally substituted by one or more hydroxyl, (di)(C₁-C₄)(alkyl)amino, carboxyl, (C₁-C₄)alkylcarbonyl, (di)(C₁-C₄)(alkyl)aminocarbonyl, such as aminocarbonyl, (C₁-C₄)alkylcarbonylamino, such as acetamido, tri(C₁-C₄)alkylammonium, cationic heterocycle, such as N—(C₁-C₄)alkylpyrrolidinium or N,N′-di(C₁-C₄)alkylpiperazinium, or cationic heteroaryl, such as N—(C₁-C₄)alkylimidazolium, groups, it being understood that the alkyl group of the alkoxy can be interrupted by one or more oxygen atoms.

Mention may be made, as examples of the couplers of the invention of formulae (I) and (la), of those of following formulae 1 to 86:

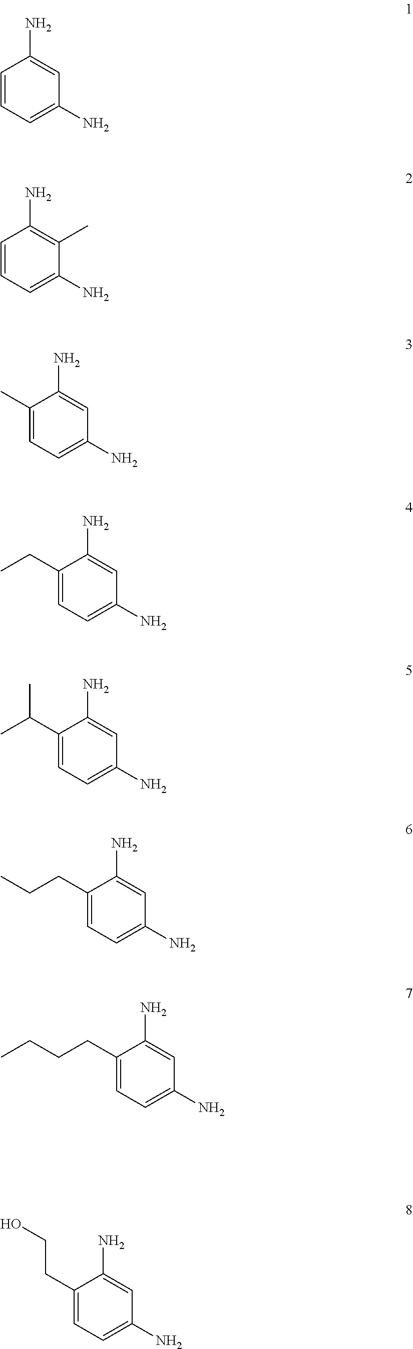

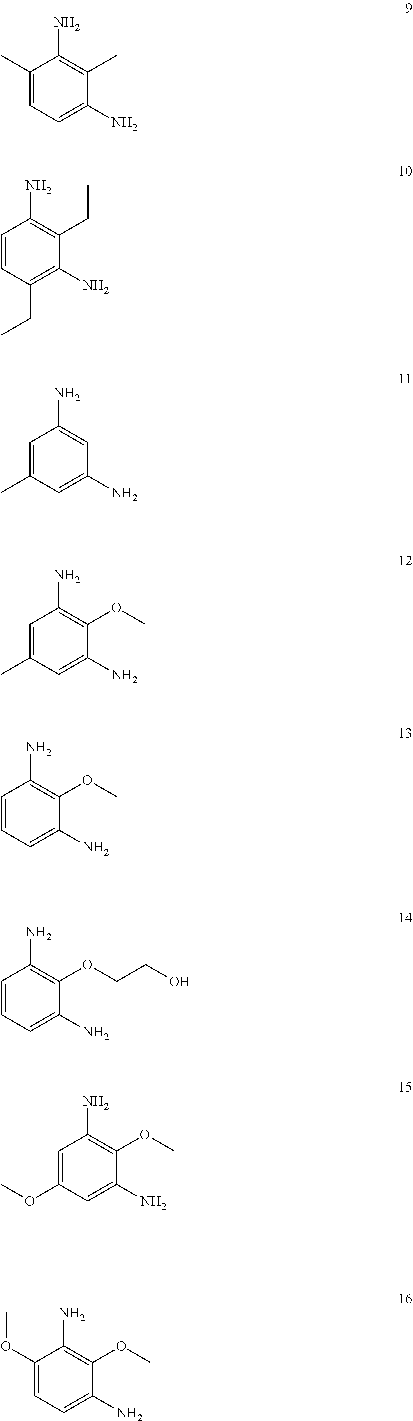

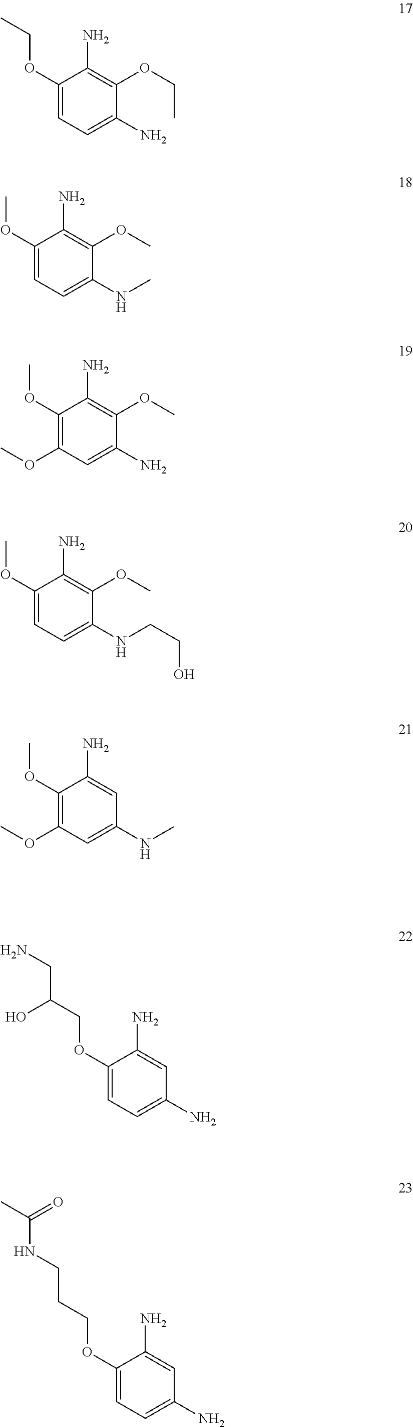

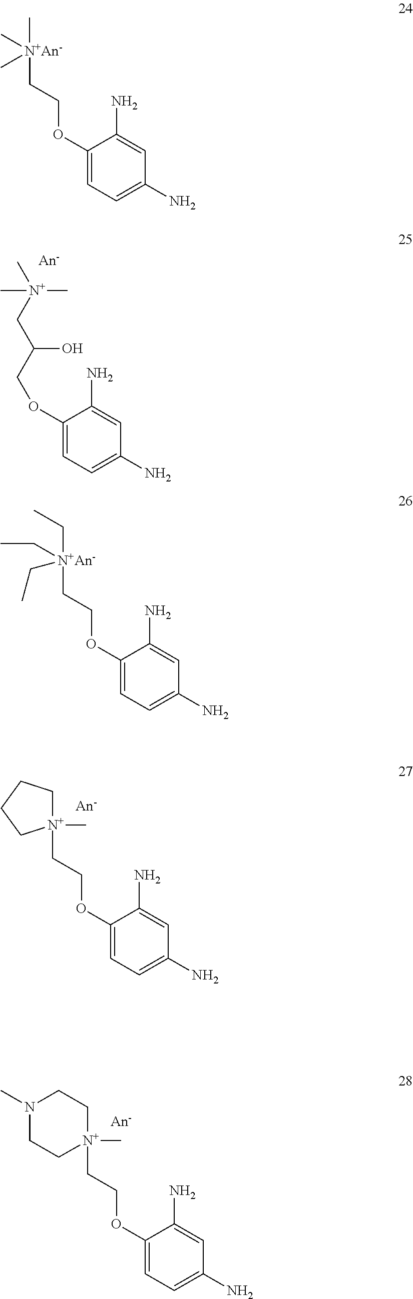

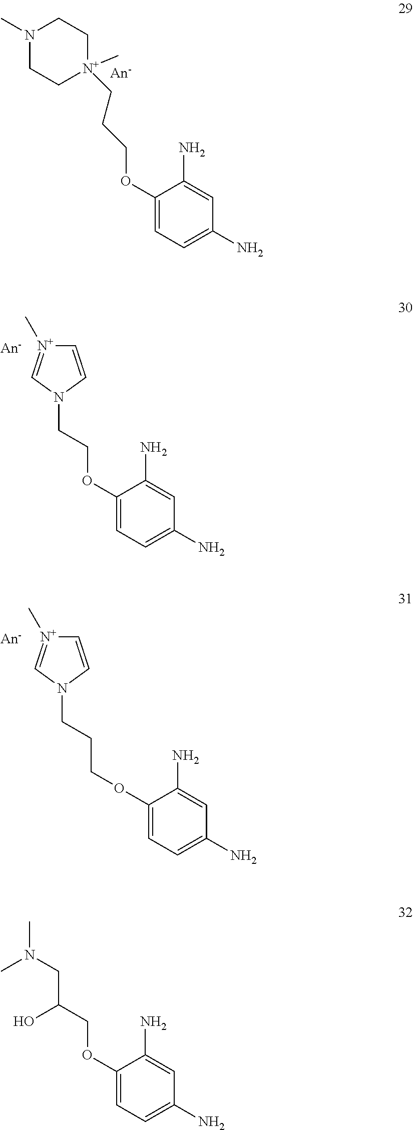 [SMILES: CN1CC[N+](C)(CCCOc2ccc(N)cc2N)CC1 ; C[n+]1ccn(CCOc2ccc(N)cc2N)c1 ; C[n+]1ccn(CCCOc2ccc(N)cc2N)c1 ; CN(C)CC(O)COc1ccc(N)cc1N]

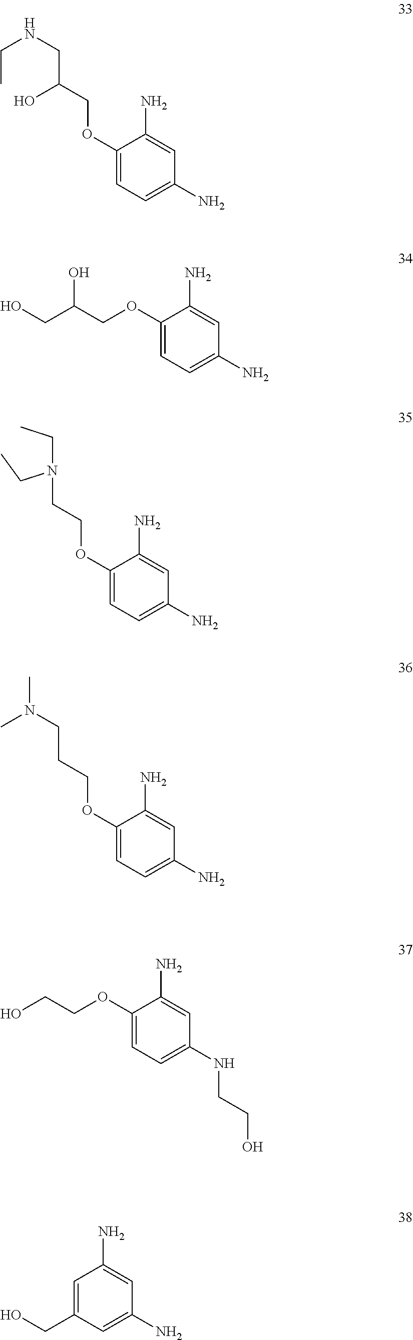 [SMILES: CCNCC(O)COc1ccc(N)cc1N ; Nc1ccc(OCC(O)CO)c(N)c1 ; CCN(CC)CCOc1ccc(N)cc1N ; CN(C)CCCOc1ccc(N)cc1N ; Nc1cc(NCCO)ccc1OCCO ; Nc1cc(N)cc(CO)c1]

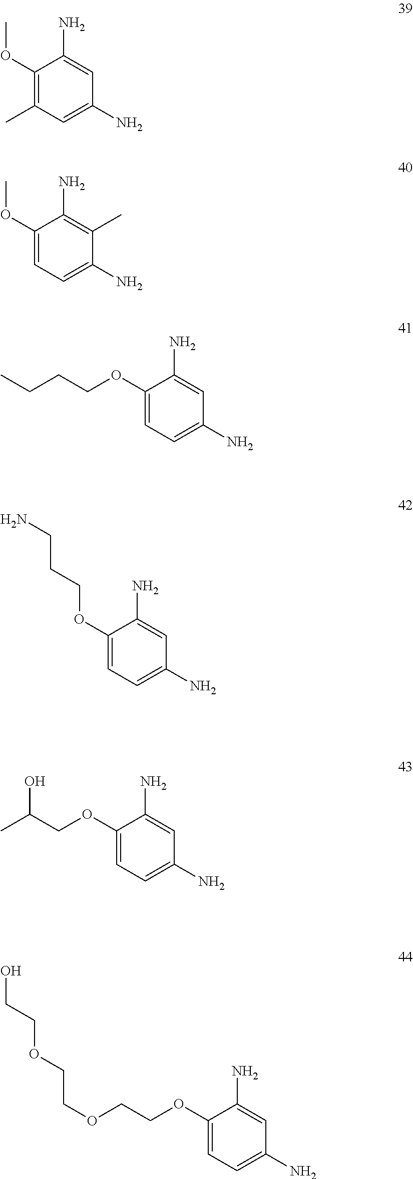 [SMILES: COc1c(C)cc(N)cc1N ; COc1ccc(N)c(C)c1N ; CCCCOc1ccc(N)cc1N ; NCCCOc1ccc(N)cc1N ; CC(O)COc1ccc(N)cc1N ; Nc1ccc(OCCOCCOCCO)c(N)c1]

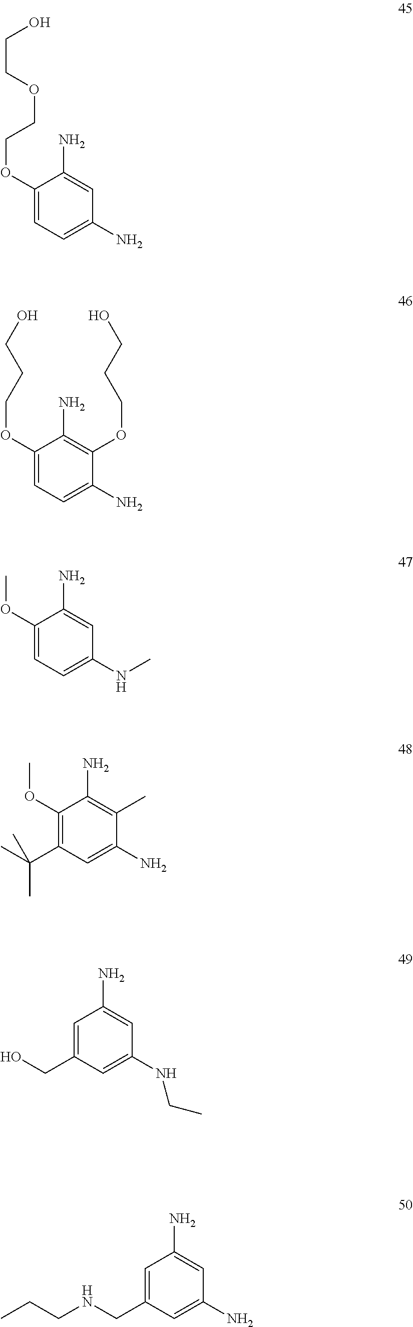

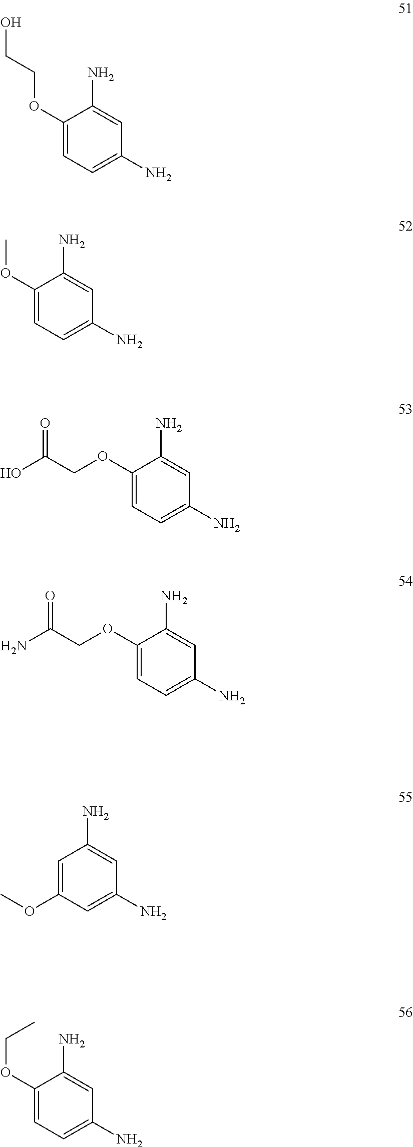

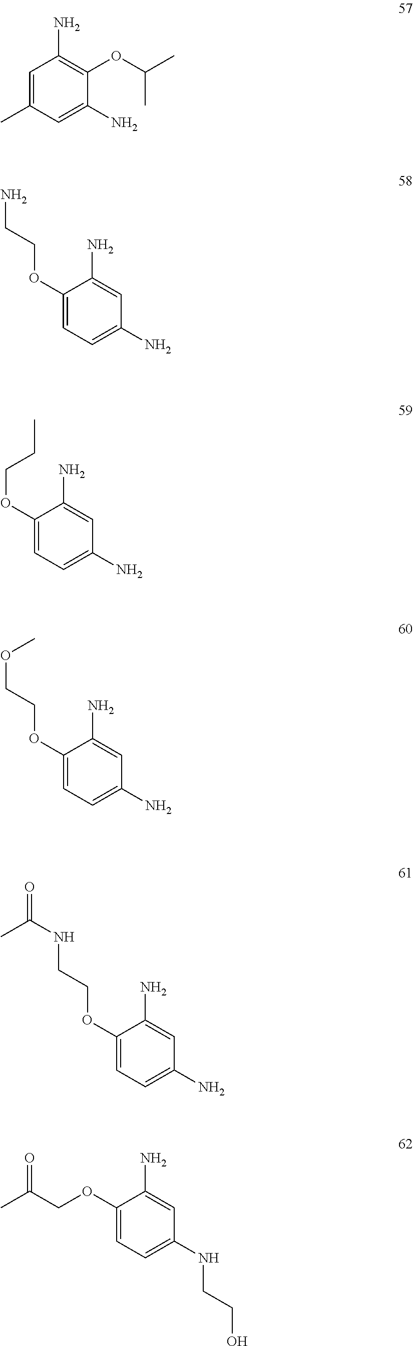

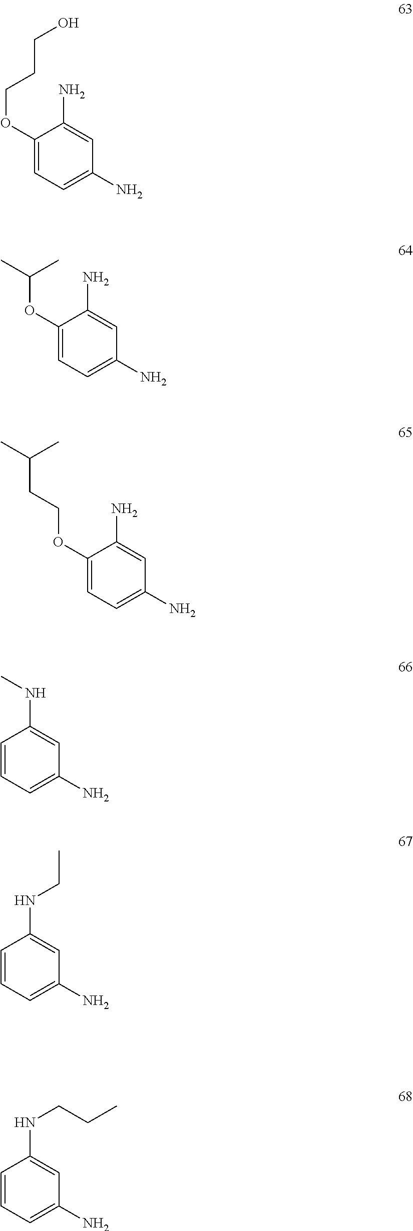

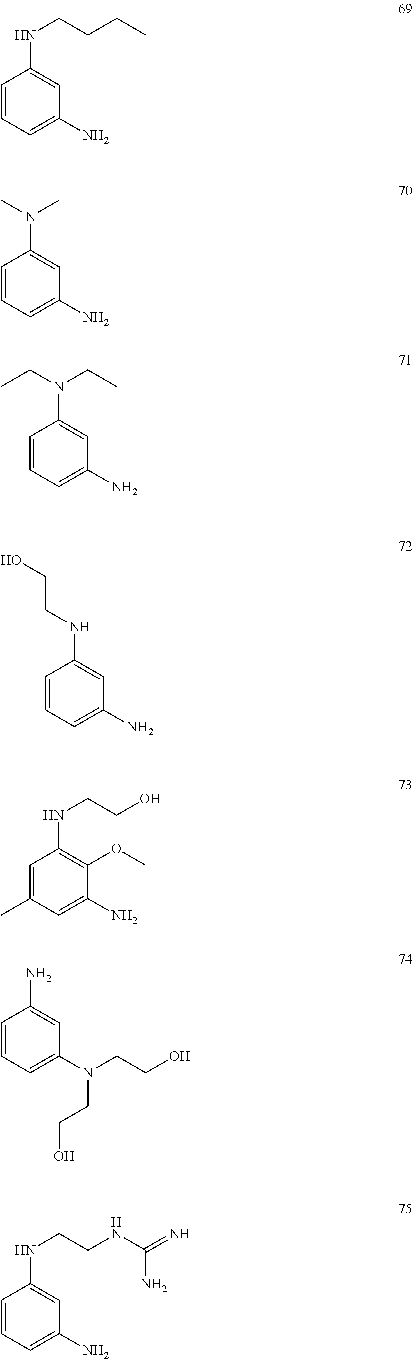

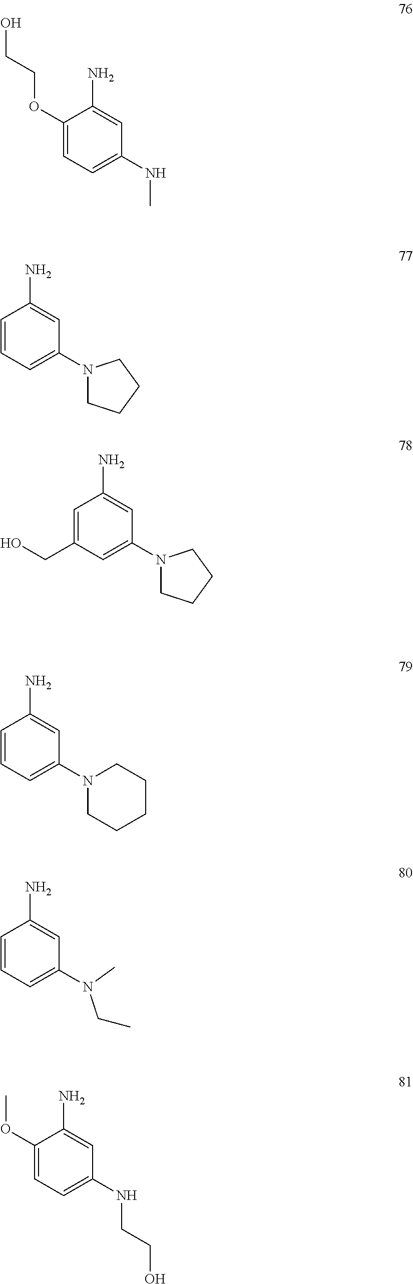

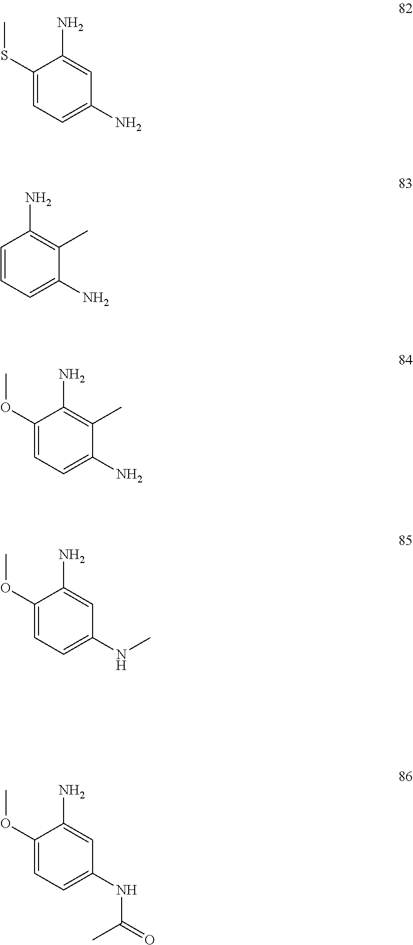

with An⁻ representing an anionic counterion, preferably of halide type, such as Cl⁻; preferably of the MPDs chosen from 3, 9, 15, 51 and 81, more especially 51.

Preferably, the dyeing agent(s) according to the invention are chosen from poly(la) compounds, in particular poly(2-(5-amino-2-methoxyphenylamino)ethanol), poly(2-(2,4-diaminophenoxy)ethanol), poly(2,4-dimethylbenzene-1,3-diamine) and poly(4-methylbenzene-1,3-diamine), especially poly(2-(2,4-diaminophenoxy)ethanol).

### ii) Radical Initiators or Chemical Oxidizing Agents

The process for the preparation of the dyeing agent(s) employs, as ingredient ii), one or more radical initiators and/or one or more chemical oxidizing agents. It is understood that the ingredient ii) is other than hydrogen peroxide in a basic or neutral medium, i.e. at a pH at 25° C. of between 7 and 11 inclusive; the ingredient ii) can represent hydrogen peroxide in a highly acidic medium, i.e. at a pH at 25° C. of less than or equal to 5, preferably of less than or equal to 3, in particular at a pH at 25° C. of between 1 and 2. According to a preferred embodiment of the invention, the ingredient ii) is other than hydrogen peroxide, whatever the pH of the medium in which it is found.

The term “and/or” is understood to mean that the ingredient comprises one or more radical initiators alone, or one or more chemical oxidizing agents alone, or comprises one or more radical initiators with one or more chemical oxidizing agents; or else that the ingredient ii) has a radical initiator nature and also a chemical oxidizing agent nature.

According to a specific embodiment of the invention, the process for the preparation of the dyeing agent(s) employs one or more ingredients ii) chosen from radical initiators.

The term “radical initiator” is understood to mean any chemical compound which can generate a radical compound under mild conditions and which induces a radical reaction (see, e.g., J. March, Advanced Organic Chemistry, Reactions, Mechanisms, and Structure, 4th Ed., John Wiley & Sons, 1992, Chap. 14 Free-radical Substitution).

In particular, these initiators comprise a weak bond, i.e. a bond with a low dissociation energy. Mention may typically be made of halogenated, azo or organic peroxide initiators. They can be generated thermally, at ambient temperature (25° C.) or at higher temperature, or photochemically.

These radical initiators are other than hydrogen peroxide.

Free-radical initiators generated thermally or from light sources are understood in particular (see, for example, Macromol. Rapid Commun., Christian Decker, 23, 1067-1093 (2002); Encyclopedia of Polymer Science and Technology, “Photopolymerization, Free Radical”, http://onlinelibrary.wiley.com/doi/10.1002/0471440264.pst490/pdf; ibid, “Photopolymerization, Cationic”, http://onlinelibrary.wiley.com/doi/10.1002/0471440264.pst491/pdf; Macromol. Symp., 143, 45-63 (1999)). Two major families may be distinguished: that
of type I, in which the radical initiators will bring about, under irradiation or heat, a unimolecular cleavage of the covalent bond to result in a free radical compound also symbolized by a “point”, and that
of type II, in which the radical initiators will result in a bimolecular reaction in which the excited state of the photoactive compounds interact with a second molecule (or co-initiator) to generate free radicals.

More particularly, the radical initiators are chosen from the compounds of formulae (V) and (VI), and also their organic or inorganic acid salts, their optical or geometrical isomers, their tautomers and their solvates, such as the hydrates:

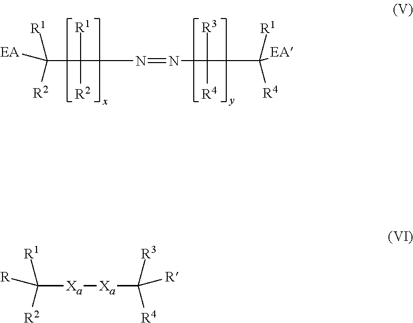

in which formulae (V) and (VI):
R¹, R², R³ and R⁴, which are identical or different, represent a hydrogen atom or a linear or branched (C₁-C₈)alkyl group which is optionally substituted, a linear or branched (C₁-C₈)alkoxy group or an optionally substituted aryl group, such as phenyl;
   or else R¹ and R² and/or R³ and R⁴ form, together with the carbon atoms which carry them, an optionally substituted 3- to 7-membered (hetero)cycloalkyl, in particular a (C₃-C₆)cycloalkyl, such as cyclohexyl;
   preferably, R¹, R², R³ and R⁴, which are identical or different, represent a hydrogen atom or a linear or branched (C₁-C₆)alkyl group;
   x and y, which are identical or different, represent an integer between 0 and 6 inclusive, and preferably x and y=0;
EA and EA′, which are identical or different, preferably identical, represent an electron-withdrawing group, which is preferably electron-withdrawing via a mesomeric effect -M, such as cyano, phosph(on)ate, sulf(on)ate, nitro or nitroso; more particularly, EA=EA′=cyano;
R and R′, which are identical or different, preferably identical, represent i) an EA or EA′ radical as defined above, or a group chosen from ii) optionally substituted and linear or branched (C₁-C₈)alkyl, iii) optionally substituted aryl or iv) optionally substituted aryl(C₁-C₈)alkyl,
or else R with R′ and/or R′ with R³ form, together with the carbon atoms which carry them, a C(X¹) group, with R² and R⁴ being as defined above, or R² and R⁴, which are identical or different, represent an R⁵—(X²)_{w}— group in which w has the value 0 or 1, R⁵ represents a hydrogen atom, a linear or branched (C₁-C₈)alkyl group, an optionally substituted (hetero)aryl group, such as phenyl, or a (hetero)cycloalkyl group which is optionally substituted, in particular by a (C₁-C₆)alkyl group, such as cyclohexyl optionally substituted by a (C₁-C₆)alkyl group, and X² is as defined below;
Xₐ, which are identical or different, represent a heteroatom chosen from oxygen and sulfur, a —C(O)—O— or —O—C(O)— group or a —O—C(O)—O— or —O—C(O)—O— group; preferably, Xₐ represent an oxygen atom;
X¹ and X², which are identical or different, represent a heteroatom chosen from oxygen, sulfur and amino N(R″) with R″ being a hydrogen atom or a linear or branched (C₁-C₆)alkyl group; preferably, X¹ and X² represent an oxygen atom;

Mention may also be made of diatomic halogen molecules, such as Cl₂, which will generate a halogen radical Cl^{▪} as radical initiator (→Cl^{▪}); azo initiators of the R—N═N—R′ type which generate the R and R′ radicals (→R^{▪} and R′^{▪}) via elimination of N₂ gas, such as AIBN (AzobislsoButyroNitrile) and ABCN (1,1′-AzoBis(CyclohexanecarboNitrile), which generate, as radical, isobutyronitrile^{▪} and cyclohexanecarbonitrile^{▪} respectively.

According to a specific embodiment of the invention, the radical initiator(s) are chosen from organic peroxides, in particular those of formula (VI) as defined above. These peroxides comprise at least one —O—O— bond which splits readily into two —O^{▪} radicals. Mention may more particularly be made of those of following general formulae:

Rₐ—C(R′ₐR′_{b})—O—O—C(R′ₐR′_{b})—R_{b} (VI₁) or

R_{c}—(O)ₐ—O—C(R′ₐR′_{b})—O—O—C(R′ₐR′_{b})—O—(O)ₛ—R_{d} (VI₂)

in which formulae (VI₁) and (VI₂):
Rₐ, R_{b}, R′ₐ and R′_{b}, which are identical or different, represent an optionally substituted (C₁-C₁₀)alkyl group, preferably (C₁-C₆)alkyl, an optionally substituted (hetero)aryl group, such as phenyl, or an optionally substituted (hetero)cycloalkyl group, such as cyclohexyl optionally substituted by (C₁-C₄)alkyl groups; or else R′ₐ and R′_{b} form, together with the carbon atom which carries them, a (thio)carbonyl group, preferably a carbonyl group;
R_{c} and R_{d}, which are identical or different, represent a hydrogen atom or a group as defined for Rₐ or R_{b}; preferably, R_{c} and R_{d} represent a hydrogen atom or a cycloalkyl group, such as cyclohexyl, optionally substituted by a (C₁-C₆)alkyl group;
Xₐ and X_{b}, which are identical or different, represent a heteroatom, such as an oxygen or sulfur atom, preferably an oxygen atom; and
s has the value 0 or 1.

According to one embodiment, the radical initiator(s) have the formula below:

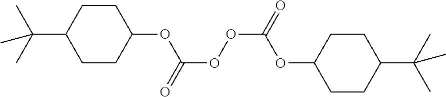 [SMILES: CC(C)(C)C1CCC(OC(=O)OOC(=O)OC2CCC(C(C)(C)C)CC2)CC1]

According to a specific embodiment of the invention, the radical initiator is AIBN (azobisisobutyronitrile). This initiator generates free radicals i) under the influence of heat at a temperature of greater than or equal to 45° C., preferably at a temperature of greater than or equal to 55° C., more particularly 60° C.; and/or ii) photochemically.

According to another specific embodiment of the invention, the radical initiator is ABDV (2,2′-azobis(2,4-dimethylvaleronitrile)). This agent can be used under thermally “milder” polymerization conditions. Preferably, when ABDV is used, the polymerization process is carried out at a temperature of greater than or equal to 28° C. and preferably at a temperature of greater than or equal to 35° C., such as 40° C.

According to another specific embodiment of the invention, the process for the preparation of the dyeing agent(s) employs one or more ingredients ii) chosen from chemical oxidizing agents.

“Chemical oxidizing agent” is understood to mean an agent other than atmospheric oxygen which has the property of being able to oxidize molecules, i.e. to remove an electron from a molecule.

According to a particularly advantageous embodiment, the ingredient(s) ii) are chosen from the persulfates of formula (VII):

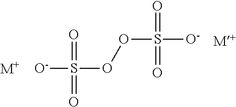

in which formula (VII) M⁺ represents an inorganic or organic cationic counterion chosen in particular from cations of alkali metals, such as sodium or potassium, or of alkaline earth metals, and ammonium R^{a}R^{b}R^{c}R^{d}N⁺, with R^{a}, R^{b}, R^{c} and R^{d}, which are identical or different, representing a hydrogen atom or a (C₁-C₆)alkyl group, such as NH₄⁺.

In particular, the ingredient(s) ii) are chosen from:
organic or inorganic persulfates of formula (VII), such as ammonium persulfates;
organic peroxides, such as di(tert-butyl) peroxide, benzoyl peroxide (BPO) or methyl ethyl ketone peroxide (MEKP);
organic peroxides, such as benzoyl peroxide, dicumyl peroxide or di(tert-butyl) peroxide;
azo compounds, such as 2,2′-azobisisobutyronitrile (AIBN) or 1,1′-azobis (cyclohexanecarbonitrile) (ABCN); and
the products of reaction:
   between hydrogen peroxide and ascorbic acid,
   between hydrogen peroxide and metal salts, in particular metal salts of transition metals, such as Fe, in particular Fe²⁺ metal salts;
   between organic peroxides, such as ammonium persulfate, and metabisulfites;
alkali metal or alkaline earth metal dichromate, such as sodium dichromate;
copper halides, in particular of Cu(II) copper, such as CuCl₂;
the product of reaction between hydrogen peroxide and ascorbic acid; and
the product of reaction between hydrogen peroxide and metal salts, such as Fe²⁺ (Fenton reaction).

Preferably, the ingredient(s) ii) are chosen from the persulfates of formula (VII) as defined above, in particular persulfates comprising organic cationic counterions, such as those for which M⁺ represents an ammonium group R^{a}R^{b}R^{c}R^{d}N⁺, with R^{a}, R^{b}, R^{c} and R^{d}, which are identical or different, representing a hydrogen atom or a (C₁-C₆)alkyl group, such as NH₄⁺.

### Process for the Preparation of the Dyeing Agent(s):

Preferably, the dyeing agent(s) of the invention are prepared by polymerization of the MPD couplers i) as defined above in the presence of radical initiators or chemical oxidizing agents ii) as defined above at a temperature of between 0° C. and 80° C. and more particularly between 0° C. and 35° C.

According to another specific preparation embodiment, dyeing agents of the invention are prepared from couplers i) as defined above and from photoactive radical initiators as ingredients ii) in the presence of one or more identical or different light sources. The said light source(s) photoirradiate by electromagnetic waves with wavelengths within the UV region up to the IR region.

“Photoirradiation with an electromagnetic wave” is understood to mean any exposure of the composition or part of the composition to a light wave during the hair dyeing method. The light spectrum can comprise wavelengths within the UV region (200-400 nm), the visible region (400-745 nm) and the infrared region (745 nm to 3 μm).

Ultraviolet radiation, visible light or infrared radiation can be used. The choice depends on the characteristic absorption of the photoactive radical initiator. If the photoactive radical initiator absorbs in the ultraviolet wavelengths, ultraviolet radiation is then used to activate it. If the photoactive radical initiators absorb in the visible wavelengths, visible radiation is then used. The absorption spectra of the photoactive radical initiators are available in the literature.

The amount of light energy varies as a function of the photoactive radical initiators. Light energy sufficient to activate the said photoactive initiator simply suffices. Sometimes daylight is sufficient. According to a specific form of the invention, the dyeing method is carried out in natural sunlight or natural daylight.

According to another process for the preparation of the dyeing agents of the invention, the source of the photoirradiation is artificial. For the lamps emitting in the UV region, mention may be made of those described in Ullmann's Encyclopedia, “Ultraviolet and Visible Spectroscopy”, 2008, Wiley-VCH Verlag GmbH & Co. KGaA, Weinheim, 10.1002/14356007.b05 383.pub2, point 3.2. For the lamps in general, mention may be made of those mentioned in Ullmann's Encyclopedia, “Lamps”, 2005, Wiley-VCH Verlag GmbH & Co. KGaA, Weinheim, 10.1002/14356007.a15 115, and Ullmann's Encyclopedia, “Photochemistry”, 2005 Wiley-VCH Verlag GmbH & Co. KGaA, Weinheim, 10.1002/14356007.a19 573, point 3.2, “light sources”.

The lamps used in the process of the invention are in particular incandescent, halogen, fluorescent, mercury or low-pressure lamps, low-pressure lamps, for example sodium or neon lamps, high-pressure lamps, for example mercury lamps, halide lamps, flash lamps, for example xenon flash lamps, fluorescent excimer lamps, such as xenon fluorescent excimer lamps, Light Emitting Diodes or LEDs of 0.01 to 1000 mW, lamps emitting black light or Wood's light, and lasers. Preferably, the artificial sources originate from mercury lamps, tungsten halogen lamps, white neon tubes, LEDs or UV lamps emitting at 254 nm or at 365 nm.

According to an advantageous embodiment of the invention, the process for the preparation of the dyeing agents employs a first stage in which the MPD couplers i) as defined above are dissolved in a liquid solution, preferably in an aqueous/alcoholic solution, then, in the second stage, one or more alkaline agents as defined above are added, in particular with aqueous ammonia, to give a basic pH preferably of between 8 and 10, and then, during a third stage, the radical initiator(s) ii) as defined above are added, in particular persulfates of formula (VII), more particularly those comprising organic cationic counterions, such as those for which M⁺ represents an ammonium group R^{a}R^{b}R^{c}R^{d}N⁺ with R^{a}, R^{b}, R^{c} and R^{d}, which are identical or different, representing a hydrogen atom or a (C₁-C₆)alkyl group, such as NH₄⁺, as are defined above. In particular, the preparation process is carried out at ambient temperature (25° C.). According to a specific embodiment, the preparation process is carried out with stirring for between 30 minutes and 2 days and preferably between 1 hour and 3 hours. According to an alternative form, during a fourth stage, the precipitate formed is filtered off, washed, preferably with water, and then dried, preferably under vacuum.

Preferably, the amounts of i) and ii) observe a weight ratio i) of the MPD coupler(s)/ii) of the radical initiator(s) or of the chemical oxidizing agent(s) of between 0.5 and 1.5, and more particularly the ratio has the value 1.

### Dyeing Agents—Coloured Oligomers or Polymers:

According to one embodiment of the invention, the dyeing agent(s) are obtained from the polycondensation i) of the MPD couplers as defined above which react with one another in the presence ii) of at least one radical initiator or of at least one chemical oxidizing agent as defined above.

More particularly, the dyeing agent(s) are chosen from poly(Ia) compounds with (la) as defined above, in particular poly(2-(5-amino-2-methoxyphenylamino)ethanol), poly(2-(2,4-diaminophenoxy)ethanol), poly(2,4-dimethylbenzene-1,3-diamine), poly(4-methylbenzene-1,3-diamine) and poly(3-methylcarbonylamino-6-methoxyaniline). Preferably, the dyeing agent(s) according to the invention are chosen from poly(Ia) compounds, in particular poly(2-(5-amino-2-methoxyphenylamino)ethanol), poly(2-(2,4-diaminophenoxy)ethanol), poly(2,4-dimethylbenzene-1,3-diamine) and poly(4-methylbenzene-1,3-diamine).

Another subject-matter of the invention is a dyeing agent capable of being obtained by chemical polycondensation: i) of MPD couplers with one another as are defined above in the presence of at least one ingredient ii) chosen from radical initiators and chemical oxidizing agents as defined above, devoid of oxidation base and devoid of hydrogen peroxide, preferably with a weight ratio of i)/ii) of between 0.5 and 1.5; more particularly, the ratio has the value 1.

Another subject-matter relates to the use of a dyeing agent, preferably a pigment, as defined above for dyeing keratinous fibres.

### The Composition of the Invention

Another subject-matter of the invention is a composition comprising at least one dyeing agent as defined above.

Preferably, the composition of the invention does not comprise an oxidation base and/or hydrogen peroxide. More preferably, the dyeing agent included in the composition is prepared by observing a molar ratio i) of the coupler(s)/ii) of the radical initiator(s) or of the chemical oxidizing agent(s) which is between 0.5 and 1.5; more particularly, the ratio has the value 1.

### The Cosmetic Medium:

“Cosmetic medium” is understood to mean a medium appropriate for the dyeing of keratinous fibres, also known as dyeing vehicle, which is a cosmetic medium generally composed of water or of a mixture of water and one or more organic solvents or of a mixture of organic solvents. Preferably, the composition comprises water and in a content in particular of between 5% and 95% inclusive, with respect to the total weight of the composition.

“Organic solvent” is understood to mean an organic substance which is capable of dissolving another substance without chemically modifying it.

### The Organic Solvents:

Mention may be made, as organic solvent, for example, of lower C₁-C₄ alkanols, such as ethanol and isopropanol, polyols and polyol ethers, such as 2-butoxyethanol, propylene glycol, propylene glycol monomethyl ether, diethylene glycol monoethyl ether or diethylene glycol monomethyl ether, and also aromatic alcohols, such as benzyl alcohol or phenoxyethanol, and their mixtures.

The organic solvents are preferably present in proportions preferably of between 0.1% and 40% by weight inclusive, with respect to the total weight of the dyeing composition, more preferably between 1% and 30% by weight approximately and more preferably still of between 5% and 25% by weight inclusive, with respect to the total weight of the composition.

The composition according to the invention comprises, in a cosmetic medium, an amount of dyeing agent as defined above generally of between 0.001% and 30% inclusive, with respect to the total weight of the composition.

Preferably, the amount of dyeing agents of the invention is between 0.01% and 5% by weight inclusive, with respect to the total weight of the composition. By way of example, the dye(s) are present in an amount of between 0.01% and 2% inclusive.

Preferably, the composition of the dyeing method of the invention is in the liquid form and comprises one or more dyeing agents as defined above.

### The Alkaline Agents:

The dyeing method can employ one or more alkaline agents. More particularly, the composition of the invention can additionally comprise one or more alkaline agents. This agent can be chosen from inorganic or organic or hybrid alkaline agents or their mixtures.

The inorganic alkaline agent(s) are preferably chosen from aqueous ammonia, alkali metal carbonates or bicarbonates, such as sodium carbonate or potassium carbonate and sodium bicarbonate or potassium bicarbonate, sodium hydroxide or potassium hydroxide, or their mixtures.

According to an advantageous embodiment of the invention, the alkaline agent(s) are organic amines, i.e. they comprise at least one substituted or unsubstituted amino group.

The organic alkaline agent(s) are preferably chosen from organic amines with a pK_{b} at 25° C. of less than 12, preferably of less than 10 and more advantageously still of less than 6. It should be noted that it concerns the pK_{b} corresponding to the functional group having the highest basicity.

Mention may be made, as hybrid compounds, of the salts of the abovementioned amines with acids, such as carbonic acid or hydrochloric acid.

The organic alkaline agent(s) are chosen, for example, from alkanolamines, oxyethylenated and/or oxypropylenated ethylenediamines, amino acids and the compounds of following formula (VIII):

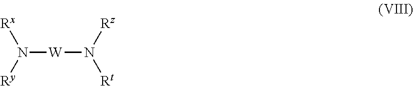

in which formula (VIII):
W is a divalent C₁-C₆ alkylene radical optionally substituted by a hydroxyl group or a C₁-C₆ alkyl radical and/or optionally interrupted by one or more heteroatoms, such as oxygen or NW;
R^{x}, R^{y}, R^{z}, R^{t} and R^{u}, which are identical or different, represent a hydrogen atom or a C₁-C₆ alkyl, C₁-C₆ hydroxyalkyl or C₁-C₆ aminoalkyl radical.

Mention may be made, as examples of such amines, of 1,3-diaminopropane, 1,3-diamino-2-propanol, spermine or spermidine.

Alkanolamine is understood to mean an organic amine comprising a primary, secondary or tertiary amine functional group and one or more linear or branched C₁-C₈ alkyl groups carrying one or more hydroxyl radicals.

The composition of the invention preferably comprises one or more alkanolamines. More preferably still, the organic amine is monoethanolamine.

In an alternative form of the invention, the composition comprises, as alkaline agent, one or more alkanolamines (preferably ethanolamine) and aqueous ammonia. In this alternative form, the alkanolamine(s) are present in a predominant amount with respect to the aqueous ammonia.

According to another alternative form, the dyeing composition comprises aqueous ammonia.

Advantageously, the composition according to the invention exhibits a content of alkaline agent(s) ranging from 0.01% to 30% by weight, preferably from 0.1% to 20% by weight and better still from 1% to 10% by weight, with respect to the weight of the said composition.

### The Adjuvants:

The composition comprising the dyeing agent(s) as defined above can also include various adjuvants conventionally used in hair dyeing compositions, such as anionic, cationic or non-ionic surfactants or their mixtures, anionic, cationic, non-ionic, amphoteric or zwitterionic polymers or their mixtures, inorganic or organic thickening agents, penetrating agents, sequestering agents, fragrances, buffers, dispersing agents, conditioning agents, such as, for example, volatile or non-volatile and modified or unmodified silicones, film-forming agents, ceramides, preservatives or opacifying agents.

The above adjuvants are generally present in an amount, for each of them, of between 0.01% and 20% by weight, with respect to the weight of the composition.

Of course, a person skilled in the art will take care to choose this or these optional additional compound(s) so that the advantageous properties intrinsically attached to the dyeing composition in accordance with the invention are not, or not substantially, detrimentally affected by the envisaged addition(s).

### The Additional Dyes:

The composition comprising the dyeing agent(s) as defined above of the method of the invention can furthermore comprise one or more additional direct dyes. These direct dyes are chosen, for example, from those conventionally used in direct dyeing, among which may be mentioned all the commonly used aromatic and/or non-aromatic dyes, such as neutral, acidic or cationic nitrobenzene direct dyes, neutral, acidic or cationic azo direct dyes, natural direct dyes, neutral, acidic or cationic quinone and in particular anthraquinone direct dyes, azine, triarylmethane or indoamine direct dyes, methines, styryls, porphyrins, metalloporphyrins, phthalocyanines, methinecyanines and fluorescent dyes.

Mention may be made, among the natural direct dyes, of lawsone, juglone, alizarin, purpurin, carminic acid, kermesic acid, purpurogallin, protocatechualdehyde, indigo, isatin, curcumin, spinulosin, apigenidin or orceins. Use may also be made of extracts or decoctions comprising these natural dyes and in particular henna-based extracts or poultices.

According to the invention, the additional direct dye(s) used according to the invention preferably represent from 0.001% to 10% by weight approximately of the total weight of the dyeing composition comprising the dyeing agent(s) as defined above and more preferably still from 0.05% to 5% by weight approximately.

### The pH:

The pH of the composition according to the invention is generally between 2 and 12 approximately and preferably between 3 and 11 approximately. It can be adjusted to the desired value by means of acidifying or basifying agents regularly used in the dyeing of keratinous fibres or alternatively using conventional buffer systems.

The pH of the composition is preferably between 6 and 11 inclusive, in particular between 7 and 10.5 inclusive and more particularly between 8 and 10 inclusive, such as 9.5.

Mention may be made, among the acidifying agents, by way of example, of inorganic or organic acids, such as hydrochloric acid, orthophosphoric acid, sulfuric acid, carboxylic acids, such as acetic acid, tartaric acid, citric acid or lactic acid, or sulfonic acids.

Mention may be made, among the alkaline agents, by way of example, of aqueous ammonia, alkali metal carbonates, alkanolamines, such as monoethanolamine, diethanolamine and triethanolamine, and other conventional alkaline agents used in cosmetics in hair dyeing as are defined above.

### Forms of the Composition:

The dyeing composition comprising the dyeing agent(s) as defined above can be provided in various formulation forms, such as in the form of liquids, lotions, creams or gels or in any other form appropriate for carrying out dyeing of keratinous fibres. It can also be packaged under pressure in an aerosol container in the presence of a propellant or in a non-aerosol container and can form a foam.

### Dyeing Methods of the Invention:

The method for dyeing keratinous substances, in particular keratinous fibres, preferably human keratinous fibres, such as the hair, of the invention employs at least one dyeing agent, preferably a dyeing agent capable of being obtained from the polycondensation i) of MPD couplers in the presence ii) of at least one radical initiator as are defined above. More specifically, the dyeing agent(s) are applied to the keratinous fibres. Preferably, the dyeing agent(s) are found in a cosmetic composition as defined above, which is applied to the keratinous fibres.

The leave-in time of the dyeing composition, i.e. the composition comprising the dyeing agent(s) as defined above, is between 5 minutes and 1 hour inclusive and preferably between 10 minutes and 40 minutes inclusive.

According to a specific embodiment of the invention, the dyeing method comprises the following stages:
1) a composition comprising at least one dyeing agent as defined above is applied to the keratinous substances; then
2) the dyeing composition is left to stand on the keratinous substances for a period of time of between 5 minutes and 1 hour inclusive and preferably of between 10 minutes and 40 minutes inclusive; and then
3) the keratinous substances are optionally shampooed with a conventional shampoo and/or rinsed and dried.

The application of the dyeing composition, i.e. the composition comprising the dyeing agent(s), is generally carried out at ambient temperature. However, it can be carried out at temperatures varying from 20 to 180° C.

The dyeing method according to the invention can be followed by shampooing with a conventional shampoo and/or the drying of the keratinous fibres.

The examples which follow serve to illustrate the invention without, however, exhibiting a limiting nature.

### DYEING EXAMPLES

### I) Synthesis of the Dyeing Agents

The compounds are analysed by HPLC/HRMS carried out on an Accela line coupled to an LTQ-Orbitrap (Thermo Fisher) high resolution mass spectrometer (HRMS).

### Compound 1

### Synthesis of poly(2,4-dimethoxybenzene-1,3-diamine)

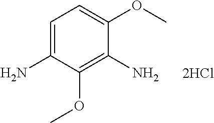

4.822 g (20 mmol) of 2,4-dimethoxybenzene-1,3-diamine dihydrochloride are dissolved in 20 ml of ethanol and 60 ml of water. The pH of this solution is brought to 9.95 by adding a few drops of 20% aqueous ammonia solution. 4.564 g (20 mmol) of ammonium persulfate in solution in 12 ml of water are subsequently added dropwise over 10 min. During the addition, the temperature rises to 35° C. and the pH decreases down to 9.2. The reaction medium is left stirring for 3 h 30 min. The precipitate is filtered off, washed with water and then dried under vacuum. Compound 1 is obtained in the form of a brown-black powder.
Ions detected:

| Retention time | | Empirical |
|---|---|---|
| Rt (min) | m/z | formula |
| 12.4 | 319⁺ | C₁₅H₁₈N₄O₄ |
| 14.4 | 303⁺ | C₁₅H₁₈N₄O₃ |
| 16.2 | 333⁺ | C₁₆H₂₀N₄O₄ |
| 17.8 | 318⁺ | C₁₅H₁₆N₅O₃ |
| 18.3 | 454⁺ | C₂₂H₂₃N₅O₆ |
| 18.4 | 452⁺ | C₂₂H₂₅N₇O₄ |
| | 500⁺ | C₂₄H₂₉N₅O₇ |
| | 615⁺ | C₃₀H₃₀N₈O₇ |
| 18.6 | 251⁺ | C₁₄H₂₂N₂O₂ |
| | 316⁺ | C₁₅H₁₇N₅O₃ |
| 18.7 | 334⁺ | C₁₆H₁₉N₃O₅ |
| 19 | 469⁺ | C₂₃H₂₈N₆O₅ |
| 19.1 | 320⁺ | C₁₅H₁₇N₃O₅ |
| 19.2 | 497⁺ | C₂₄H₂₈N₆O₆ |
| 19.4 | 467⁺ | C₂₃H₂₆N₆O₅ |
| 19.7 | 433⁺ | C₂₂H₂₀N₆O₄ |
| | 569⁺ | C₂₉H₂₈N₈O₅ |
| 20.1 | 603⁺ | C₃₀H₃₄N₈O₆ |
| 20.7 | 401⁺ | C₂₁H₂₈N₄O₄ |

### Compound 2

### Synthesis of poly(2-(5-amino-2-methoxyphenylamino)ethanol)

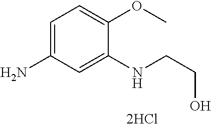

12 ml of water and 12 ml of ethanol are added to 1.020 g (4 mmol) of 2-(5-amino-2-methoxyphenylamino)ethanol dihydrochloride; the pH of this solution is adjusted to 9.5-10 with 20% aqueous ammonia. A solution of 913 mg (5 mmol) of ammonium persulfate in 2 ml of water is then added dropwise. The reaction mixture is left stirring for 3 h and the precipitate formed is filtered off and washed with water/ethanol. Compound 2 is obtained in the form of a black powder.
Ions detected:

| Rt (min) | m/z | Empirical formula |
|---|---|---|
| 14.7 | 346⁺ | C₁₇H₂₃N₅O₃ |
| 17.1 | 285⁺ | C₁₅H₁₆N₄O₂ |
| 17.9 | 344⁺ | C₁₇H₂₁N₅O₃ |
| 18.2 | 480⁺/2402⁺ | C₂₅H₂₉N₅O₅ |
| 19.1 | 405⁺ | C₂₂H₂₀N₄O₄ |
| 19.9 | 405⁺ | C₂₂H₂₀N₄O₄ |

### Compound 3

### Synthesis of poly(2-(2,4-diaminophenoxy)ethanol)

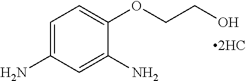 [SMILES: Nc1ccc(OCCO)c(N)c1]

24.1 g (0.1 mol) of 2-(2,4-diaminophenoxy)ethanol dihydrochloride are dissolved in 300 ml of water. The pH is subsequently brought to 10.2 by adding approximately 60 ml of a 20% aqueous ammonia solution. 22.8 g (0.1 mol) of ammonium persulfate in solution in 50 ml of water are then added dropwise over 40 minutes. During the addition, the temperature rises to 31° C. and the pH decreases from 10.2 to 9.5. The reaction medium is left stirring for 3 h 30 min. The precipitate formed is filtered off and washed with water. After drying, compound 3 is obtained in the form of a black powder.
Ions detected:

| Rt (min) | m/z | Empirical formula |
|---|---|---|
| 6.6 | 289⁺ | C₁₄H₁₆N₄O₃ |
| 9.1 | 333⁺ | C₁₆H₂₀N₄O₄ |
| 9.4 | 288⁺ | C₁₄H₁₇N₅O₂ |
| 14.2 | 271⁺ | C₁₄H₁₄N₄O₂ |
| 15.8 | 438⁺ | C₂₂H₂₃N₅O₅ |
| 17 | 437⁺ | C₂₂H₂₄N₆O₄ |
| 17.9 | 586⁺ | C₃₀H₃₁N₇O₆ |
| | 435⁺ | C₂₂H₂₂N₆O₄ |
| | 375⁺ | C₂₀H₁₈N₆O₂ |

### Compound 4

### Synthesis of poly(2,4-dimethylbenzene-1,3-diamine)

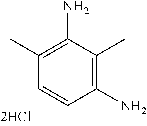

1045.5 mg (5 mmol) of 2,4-dimethylbenzene-1,3-diamine dihydrochloride are dissolved in 10 ml of ethanol and 10 ml of water. The pH is subsequently brought to 10 by adding approximately 6 ml of a 20% aqueous ammonia solution. 1141 mg (5 mmol) of ammonium persulfate in solution in 3 ml of water are subsequently added dropwise over 7 min. During the addition, the temperature rises to 28.5° C. and the pH changes from 10 to 9.47. The reaction medium is left stirring for 6 h 30 min. The precipitate is filtered off and the precipitate formed is washed with water. After drying, compound 4 is obtained in the form of a brown powder.
Ions detected:

| Rt (min) | m/z | Empirical formula |
|---|---|---|
| 10 | 269⁺ | C₁₆H₂₀N₄ |
| 12.1 | 285⁺ | C₁₆H₂₀N₄O |
| 17.2 | 403⁺/2022⁺ | C₂₄H₃₀N₆ |
| 18.4 | 269⁺ | C₁₆H₂₀N₄ |
| 19 | 267⁺ | C₁₆H₁₈N₄ |
| 19.2 | 267⁺ | C₁₆H₁₈N₄ |
| 20.3 | 270⁺ | C₁₆H₂₀N₃O |
| | 401⁺ | C₂₄H₂₈N₆ |
| 21.2 | 269⁺/1352⁺ | C₁₆H₂₀N₄ |
| 24 | 504⁺ | C₃₁H₃₃N₇ |

### Compound 5

### Synthesis of poly(4-methylbenzene-1,3-diamine)

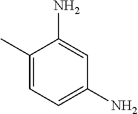

610.85 mg (5 mmol) of 4-methylbenzene-1,3-diamine are dissolved in 10 ml of ethanol and 3 ml of water in a round-bottomed flask. The pH of this solution is brought to 9.9 with a 20% aqueous ammonia solution. 1141 mg (5 mmol) of ammonium persulfate in solution in approximately 3 ml of water are subsequently added dropwise over 10 min. During the addition, the temperature rises to 33° C. and the pH decreases from 10.2 to 3.1. The reaction medium is left stirring for 3 h 30 min, then the pH is brought to 6.7 with 20% NH₃ and the solid formed is filtered off, washed with water and dried. After drying, compound 5 is obtained in the form of a brown powder.
Ions detected:

| Rt (min) | m/z | Empirical formula |
|---|---|---|
| 3.1 | 243⁺ | C₁₄H₁₈N₄ |
| 6 | 346⁺ | C₂₁H₂₃N₅ |
| 6.4 | 346⁺ | C₂₁H₂₃N₅ |
| 10.7 | 242⁺ | C₁₄H₁₅N₃O |
| 13.4 | 344⁺ | C₂₁H₂₁N₅ |
| 15.7 | 239⁺ | C₁₄H₁₄N₄ |
| 18.1 | 270⁺ | C₁₆H₁₉N₃O |
| 18.6 | 424⁺ | C₂₀H₂₅N₉O₂ |

### Compound 6

### Synthesis of poly(N-(3-amino-4-methoxyphenyl)acetamide)

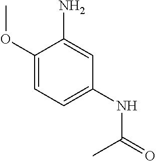

901 mg (5 mmol) of N-(3-amino-4-methoxyphenyl)acetamide are dissolved in 15 ml of ethanol and 5 ml of water. The pH is brought to 10.3 by adding 0.3 ml of a 20% aqueous ammonia solution. 1141 mg (5 mmol) of ammonium persulfate in solution in 3 ml of water are subsequently added dropwise over 10 min. The reaction medium is left stirring for 1 h 15 min. The pH is neutralized. The product is filtered off and washed with water. After drying, 415 mg of compound 6 are obtained in the form of a black powder.

### II) Evaluation on Keratinous Fibres

### Protocol A:

A formulation vehicle is prepared by mixing, respectively:

0.5 g of benzoic acid

5 g of benzyl alcohol

15 g of ethanol

water until 100 g of mixture are obtained

20% aqueous ammonia until a pH of 9.5 is obtained.

0.5 g of coloured oligomers is added to this formulation vehicle and the resulting mixture is then applied to natural locks comprising 90% white hairs (liquor/hair ratio 5/1) at 20° C. for 30 minutes. The locks are subsequently washed and shampooed.

### Protocol B:

A formulation vehicle is prepared by mixing, respectively:

5 g of a 30% aqueous oleocetyldimethylhydroxyethylammonium chloride solution

5 g of benzyl alcohol

15 g of ethanol

water until 100 g of mixture are obtained

20% aqueous ammonia until a pH of 9.5 is obtained.

0.5 g of coloured oligomers is added to this formulation vehicle and the resulting mixture is then applied to natural locks comprising 90% white hairs (liquor/hair ratio 5/1) at 20° C. for 30 minutes. The locks are subsequently washed and shampooed.

### Calculation of the Colour Variation ΔE*

The coloration build-up (ΔE*) also called uptake was evaluated in the CIE L* a* b* system. In this L* a* b* system, L* represents the intensity of the colour, a* indicates the green/red colour axis and b* indicates the blue/yellow colour axis. The lower the value of L*, the darker or more intense the colour.
The value of ΔE* was calculated from the values of L*a*b* according to equation (i) below:

ΔE_{ab}*=\sqrt{(L*−Lₒ*)²+(a*−aₒ*)²+(b*−bₒ*)²}

The coloration build-up (ΔE*) was calculated on the locks of untreated hair (L₀*, aₒ* and bₒ*) and on locks of dyed hair (L*, a* and b*).
The greater the value of ΔE*, the better the coverage of the fibres.

| | Pro- | | | | Buildup |
|---|---|---|---|---|---|
| Oligomer precursors | tocol | L | a | b | (ΔE) |
| 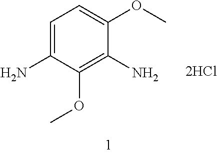 | B | 26.2 | 2 | 2.3 | 39.8 |
| 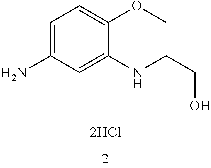 | A | 40.2 | 0.6 | 5 | 18.8 |
| 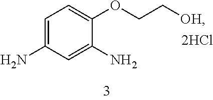 [SMILES: Nc1ccc(OCCO)c(N)c1] | A | 24.5 | 0.48 | 2.82 | 32.07 |
| 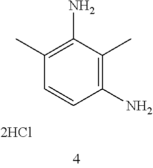 | A | 48.1 | 4.7 | 10.5 | 16.8 |
| 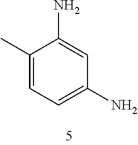 | A | 40.8 | 4.5 | 14.1 | 23.3 |

### III Comparative Essays: Oligomers of 2-(2,4-Diaminophenoxy)Ethanol with Ammonium Persulfate (Compound 3) Vs Attempt to Obtain Oligomers with Hydrogen Peroxide

As mentioned herein before oligomers of poly(2-(2,4-diaminophenoxy)ethanol), compound 3, is obtained with ammonium persulfate.
Attempt to obtain oligomers from 2-(2,4-diaminophenoxy)ethanol with hydrogen peroxide: compound 3′.
24.1 g (0.1 mol) of 2-(2,4-diaminophenoxy)ethanol dihydrochloride are dissolved in 300 ml of water. The pH is subsequently brought to 10.2 by adding approximately 60 ml of a 20% aqueous ammonia solution. 22.8 g (0.1 mol) of hydrogen peroxide at 6% (10 ml of hydrogen peroxide at 30%+40 ml of water−0.09 mol) in solution in 50 ml of water are then added dropwise over 40 minutes. During the addition, the temperature rises to 31° C. and the pH=10 at the end of the addition. The reaction medium is left stirring for 3 h 30 min. No precipitate is formed.
A natural lock comprising 90% white hairs is then treated directly with the reaction mixture (liquor/hair ratio 5/1) at 20° C. for 30 minutes. The lock is subsequently washed and shampooed.
The L measured for 3′ (comparative) is 32 vs. L=24.5 for compound 3 (invention). In spite of a higher concentration of potential oligomers present in the comparative composition (5.9 g % vs. 0.5 g % invention), color obtained after treatment of lock with compound according to the invention is a significantly more intensive than the one obtained with the comparative.

1.-16. (canceled)

17. A method for dyeing a keratinous substance, the method comprising:
applying to the keratinous substances at least one dyeing agent obtained from the polycondensation between i) at least two meta-phenylenediamine couplers which react with one another in the presence of ii) at least one ingredient chosen from radical initiators and chemical oxidizing agents.

18. The method according to claim 17, wherein the meta-phenylenediamine couplers are chosen from those of formula (I) or the salts of inorganic or organic acids or bases thereof, the optical isomers or geometrical isomers thereof, the tautomers and/or solvates thereof, or the hydrates thereof: 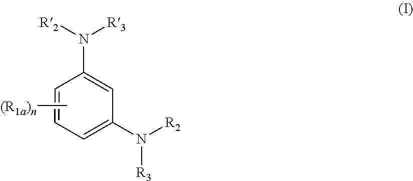
wherein in formula (I):
R₁ₐ is chosen from a halogen atom or a group chosen from:
1) (C₁-C₈)alkyl groups, optionally substituted by at least one halogen atom or at least one group chosen from hydroxyl, (di)(C₁-C₆)(alkyl)amino, —N(R)—C(O)—NR′₂, (C₁-C₆)alkoxy, or (C₁-C₆)alkylthio groups;
2) (C₁-C₈)alkoxy groups, optionally substituted by at least one halogen atom or at least one group chosen from hydroxyl, (di)(C₁-C₄)(alkyl)amino, carboxyl, R—C(O)—N(R′)—, R—S(O)₂—N(R′)—, RR′N—C(O)—, pyrrolidino, piperidino, N′—(C₁-C₄)(alkyl)-N-piperazino, morpholino, N—(C₁-C₄)alkylpyrrolidinium, N—(C₁-C₄)alkylpiperidinium, N,N′-di(C₁-C₄)alkylpiperazinium, N—(C₁-C₄)alkylmorpholinium, N—(C₁-C₄)alkylimidazolium and tri(C₁-C₄)alkylammonium groups, with R and R′, which are identical or different, chosen from a hydrogen atom or a (C₁-C₄)alkyl group, with the proviso that the alkyl group of the alkoxy is optionally interrupted by at least one heteroatom or oxygen;
3) (C₁-C₈)alkylthio groups, optionally substituted by at least one hydroxyl or R—C(O)—N(R′)— group, with R and R′ as defined above;
4) R—C(O)—N(R′)—, with R and R′ as defined above;
5) (C₁-C₄)alkoxycarbonyl;
6) carboxyl;
7) —SO₃H;
8) RR′N—C(O)—, with R and R′ as defined above;
9) (di)(C₁-C₄)(alkyl)amino; or
10) R—O—C(O)—, with R as defined above;
R₂, R₃, R′₂ and R′₃, which are identical or different, are chosen from:
i) a hydrogen atom,
ii) a (C₁-C₈)alkyl group optionally substituted by at least one of the following groups:
a) hydroxyl,
b) R′₂—(W′)ₚ—C(W)—(W″)_{q}—, aminocarbonylamino, or guanidino with p and q=0 or 1, R′₂ chosen from a hydrogen atom or a group chosen from (C₁-C₆)alkyl, W representing an oxygen or sulfur atom or an amino group N(R″), and W′ and W″ chosen from an oxygen or sulfur atom or an N(R′″) group with R″ and R′″, which are identical or different, chosen from a hydrogen atom or a (C₁-C₄)alkyl group,
c) (di)(C₁-C₆)(alkyl)amino, or
d) (C₁-C₈)alkylthio; or
iii) an R′₂—(W′)ₚ—C(W)—(W″)_{q}— group as defined above;
or R₂ with R₃ and/or R′₂ with R′₃ form, together with the nitrogen atom which carries them, a 5- to 7-membered monocyclic heterocycle, which is optionally substituted by at least one (C₁-C₆)alkyl group; (C₁-C₆)(alkyl)piperidino; or (di)azepano;
n has the value 0, 1, 2 or 3;
with the proviso that, when the compound of formula (I) comprises at least one cationic substituent, the compound comprises as many anionic counterions as cationic charges in order to achieve the electrical neutrality of the compound of formula (I).

19. The method according to claim 17, wherein the meta-phenylenediamine couplers are chosen from those of formula (Ia) or the salts of inorganic or organic acids or bases thereof, the optical isomers or geometrical isomers thereof, the tautomers and/or solvates thereof, or the hydrates thereof: 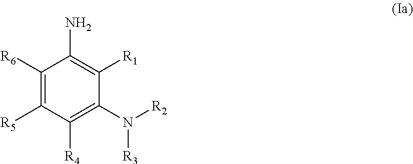
wherein in formula (Ia):
R₂ and R₃, which are identical or different, are chosen from:
i) a hydrogen atom;
ii) a (C₁-C₆)alkyl group optionally substituted by at least one group chosen from:
a) hydroxyl,
b) urea —N(H)—C(O)—NH₂,
c) guanidino —N(H)—C(NH)—NH₂,
d) (di)(C₁-C₄)(alkyl)amino, or
e) (C₁-C₄)alkylthio;
iii) a (C₁-C₄)alkylcarbonyl group, or
iv) an aminocarbonyle H₂N—C(O)— group;
or R₂ and R₃ form, together with the nitrogen atom which carries them, a saturated heterocycle;
R₁, R₄, R₅, and R₆, which are identical or different, are chosen from:
i) a hydrogen or halogen atom,
ii) a (C₁-C₆)alkyl radical optionally substituted by at least one atom or group chosen from:
a) halogen,
b) hydroxyl,
c) (di)(C₁-C₆)(alkyl)amino, or
d) (C₁-C₆)alkylthio,
iii) (C₁-C₆)alkoxy groups, optionally substituted by at least one halogen atom or at least one group chosen from hydroxyl, (di)(C₁-C₄)(alkyl)amino, carboxyl, (C₁-C₆)alkylcarbonyl, R—C(O)—N(R′)—, R—S(O)₂—N(R′)—, RR′N—C(O)—, cationic heterocycle or cationic heteroaryl, N—(C₁-C₄)alkylpyrrolidinium, N—(C₁-C₄)alkylpiperidinium, N,N′-di(C₁-C₄)alkylpiperazinium, N—(C₁-C₄)alkylmorpholinium or N—(C₁-C₄)alkylimidazolium, or tri(C₁-C₄)alkylammonium groups, with R and R′, which are identical or different, chosen from a hydrogen atom or a (C₁-C₄)alkyl group, with the proviso that the alkyl group of the alkoxy is optionally interrupted by at least one oxygen atom;
iv) (C₁-C₆)alkylthio groups, optionally substituted by at least one group chosen from hydroxyl, (di)(C₁-C₄)(alkyl)amino or R—C(O)—N(R′)—, with R and R′ as defined above;
v) R—C(O)—N(R′)—, with R and R′ as defined above;
vi) R—O—C(O)— with R as defined above;
vii) carboxyl;
viii) SO₃H; or
ix) aminocarbonyl;
with the proviso that, when the compound of formula (Ia) comprises at least one cationic substituent, the compound comprises as many anionic counterions as cationic charges in order to achieve the electrical neutrality of the compound of formula (Ia).

20. The method according to claim 19, wherein the couplers are chosen from those of formula (Ia) in which:
R₁ is chosen from a hydrogen atom or a group chosen from (C₁-C₆)alkyl or (C₁-C₆)alkoxy optionally substituted by a hydroxyl group;
R₂ and R₃, which are identical or different, are chosen from i) a hydrogen atom; ii) a (C₁-C₆)alkyl group optionally substituted by a group chosen from hydroxyl or guanidino; or iii) a (C₁-C₄)alkylcarbonyl group or acetyl CH₃—C(O)—;
or R₂ and R₃ form, together with the nitrogen atom which carries them, a saturated heterocycle chosen from pyrrolidino, piperidino, or morpholino;
R₄ represents a hydrogen atom;
R₅ represents a hydrogen atom or a group chosen from (C₁-C₆)alkyl optionally substituted by a group chosen from hydroxyl, (di)(C₁-C₄)(alkyl)amino, or (C₁-C₆)alkoxy;
R₆ is chosen from a hydrogen atom or a group chosen from:
(C₁-C₆)alkyl, or
(C₁-C₆)alkoxy optionally substituted by at least one hydroxyl, (di)(C₁-C₄)(alkyl)amino, carboxyl, (C₁-C₄)alkylcarbonyl, (di)(C₁-C₄)(alkyl)aminocarbonyl, such as aminocarbonyl, (C₁-C₄)alkylcarbonylamino, such as acetamido, tri(C₁-C₄)alkylammonium, cationic heterocycle, N—(C₁-C₄)alkylpyrrolidinium, N,N′-di(C₁-C₄)alkylpiperazinium, cationic heteroaryl, or N—(C₁-C₄)alkylimidazolium, group, with the proviso that the alkyl group of the alkoxy can be interrupted by at least one oxygen atom.

21. The method according to claim 17, wherein the meta-phenylenediamine couplers are chosen from those of formulae 1 to 86, and also the organic or inorganic acid salts thereof, the optical or geometrical isomers thereof, the tautomers or solvates thereof, or the hydrates thereof: 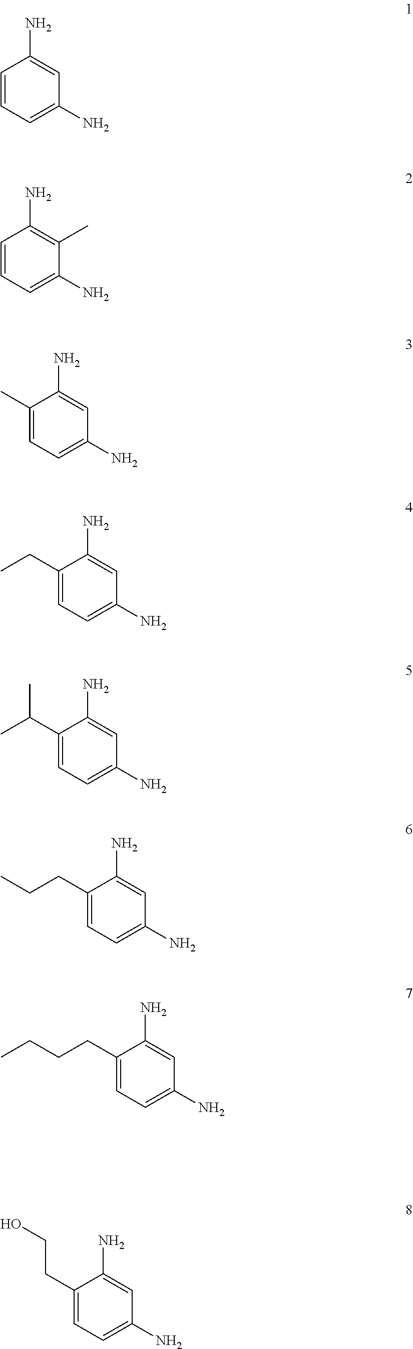 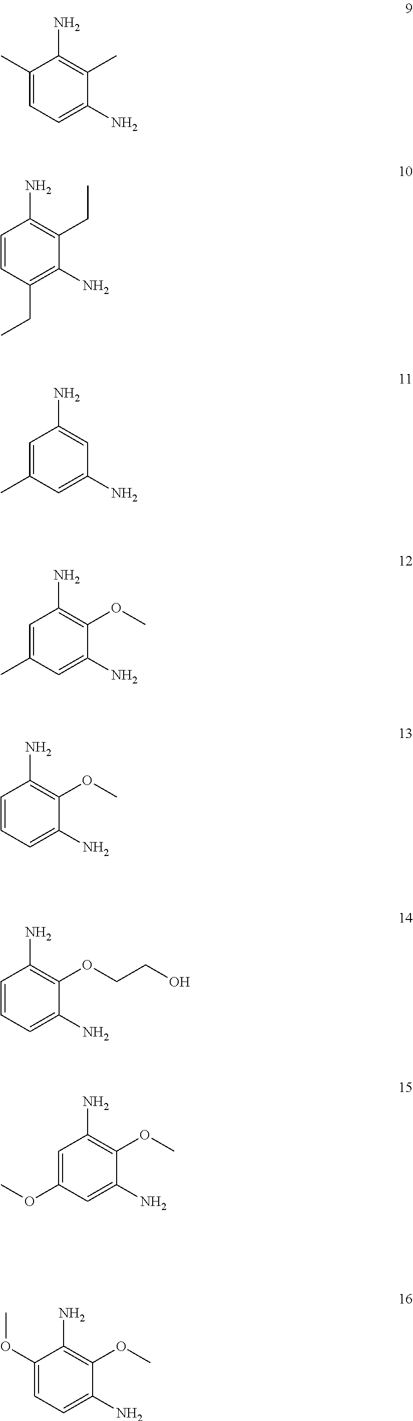 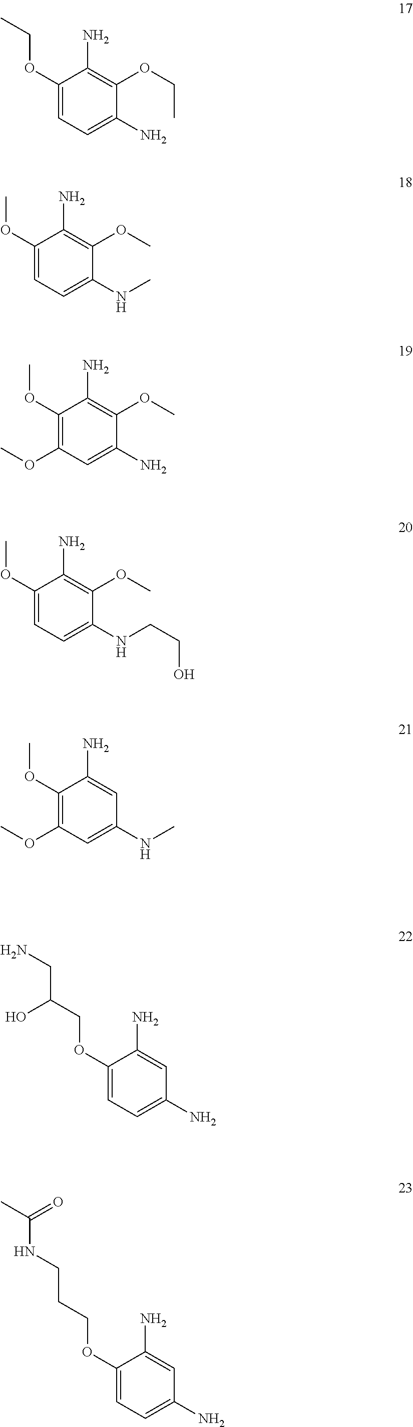 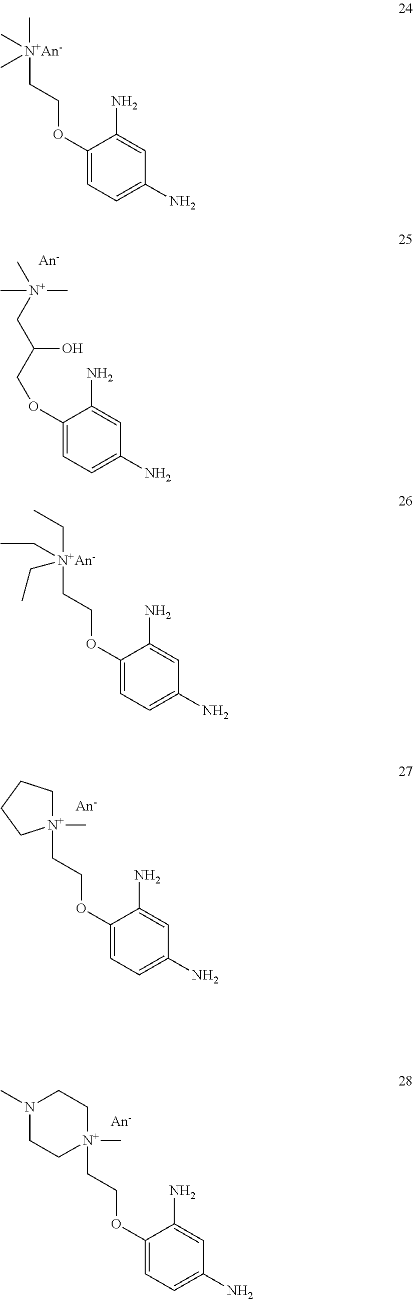 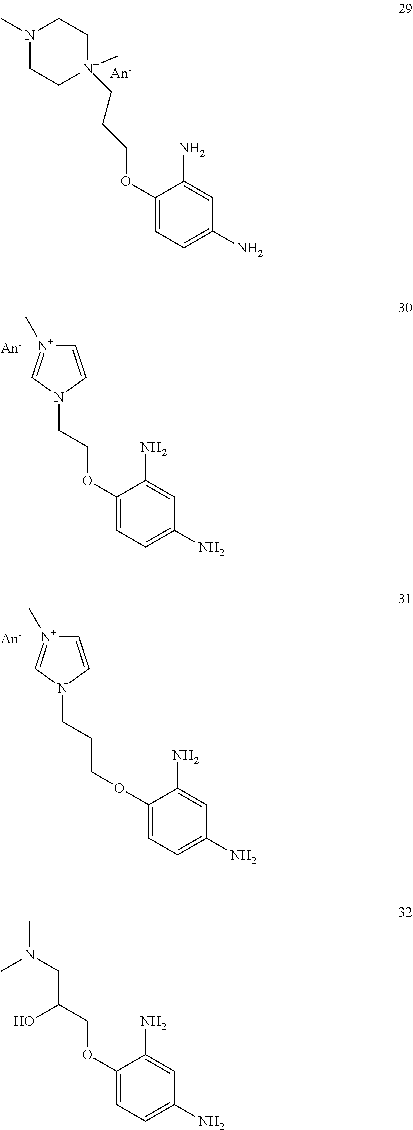 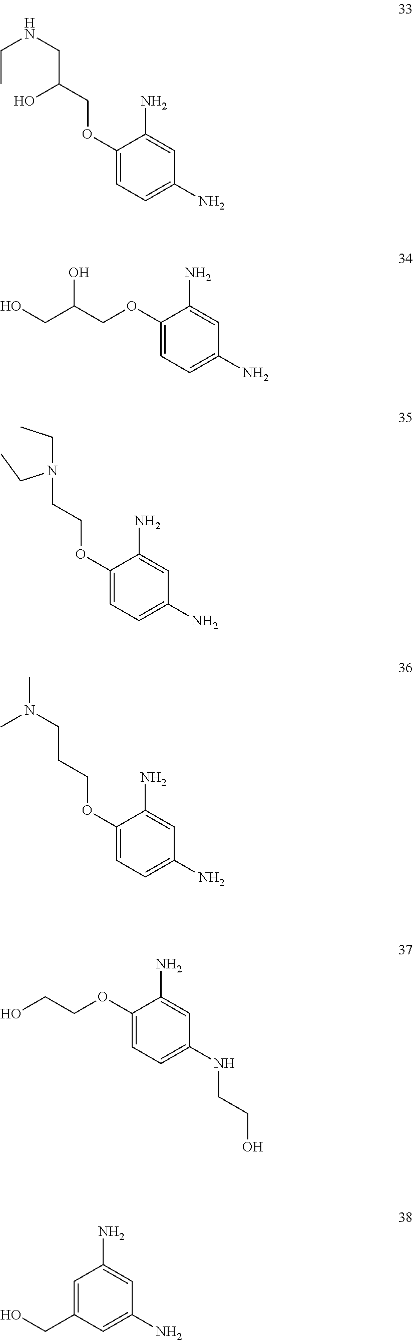 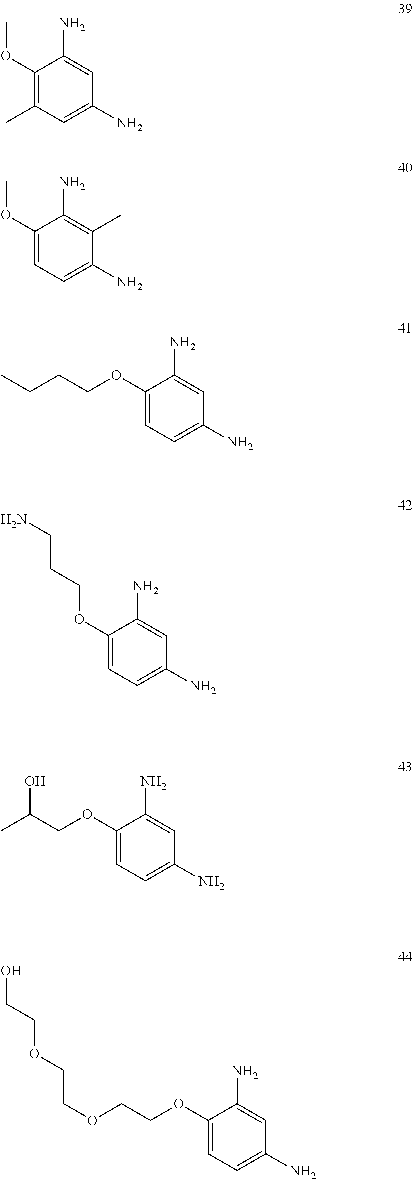 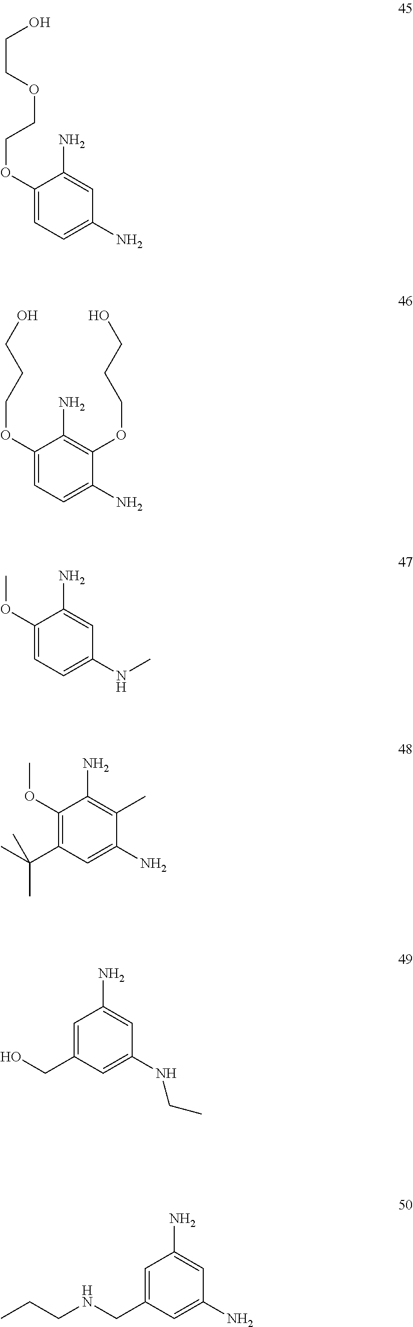 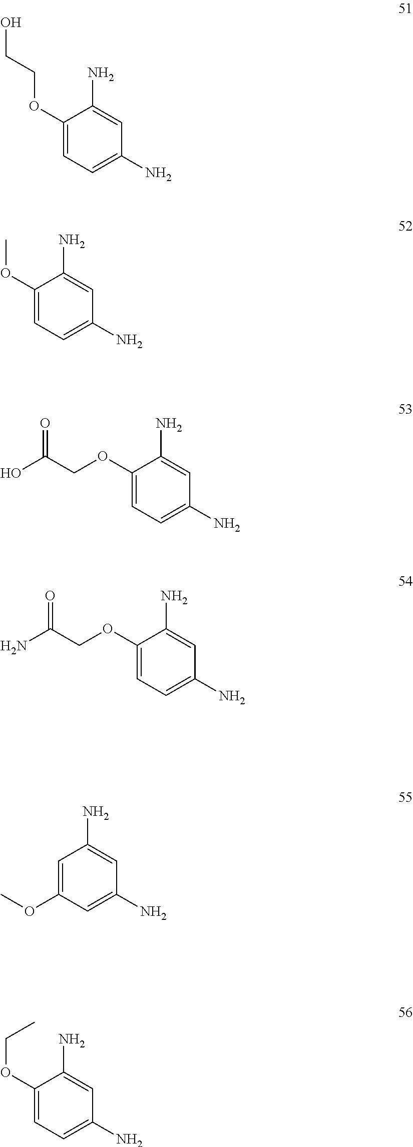 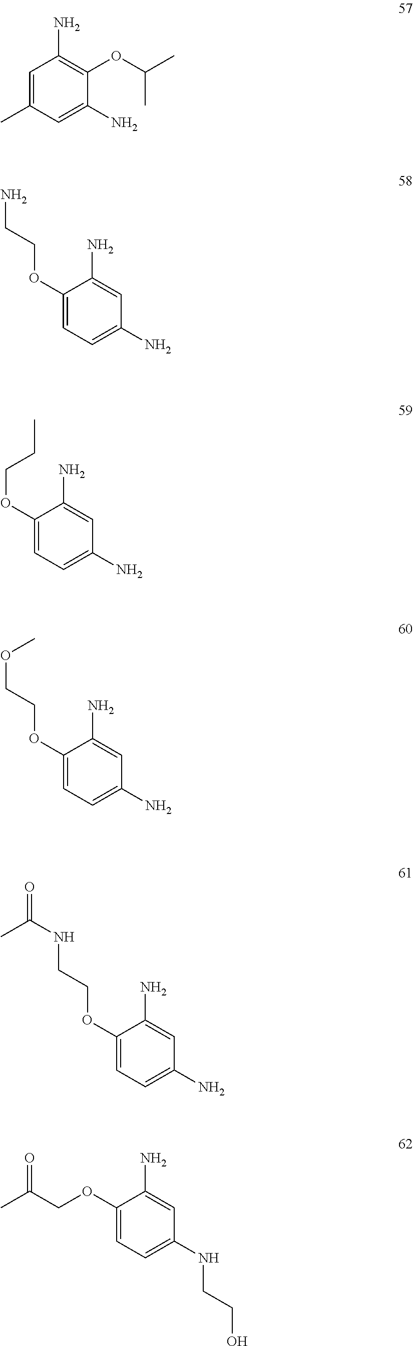 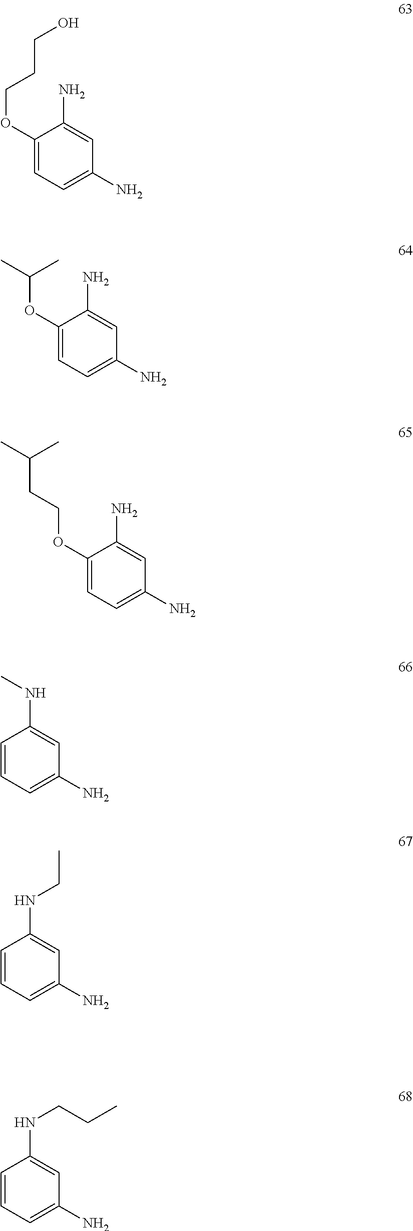 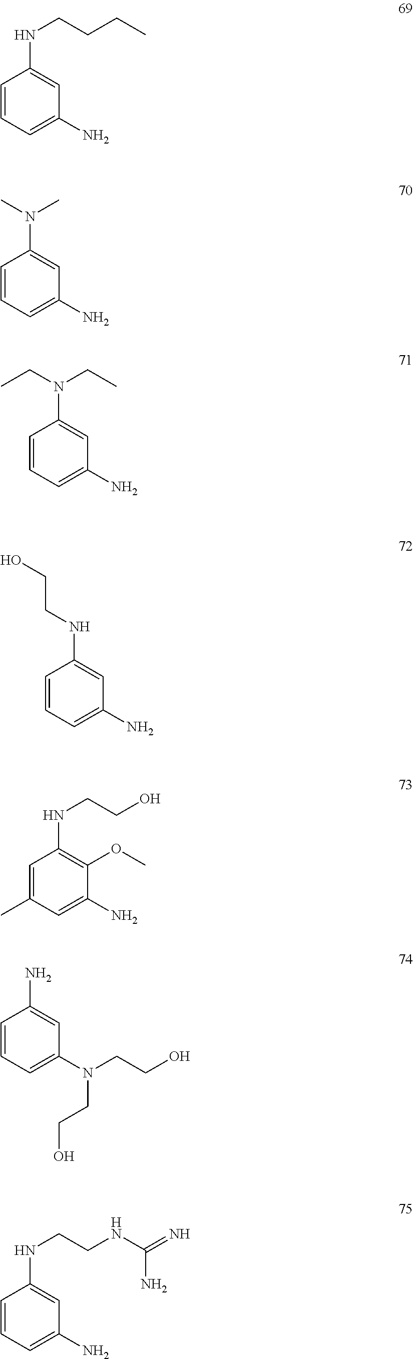 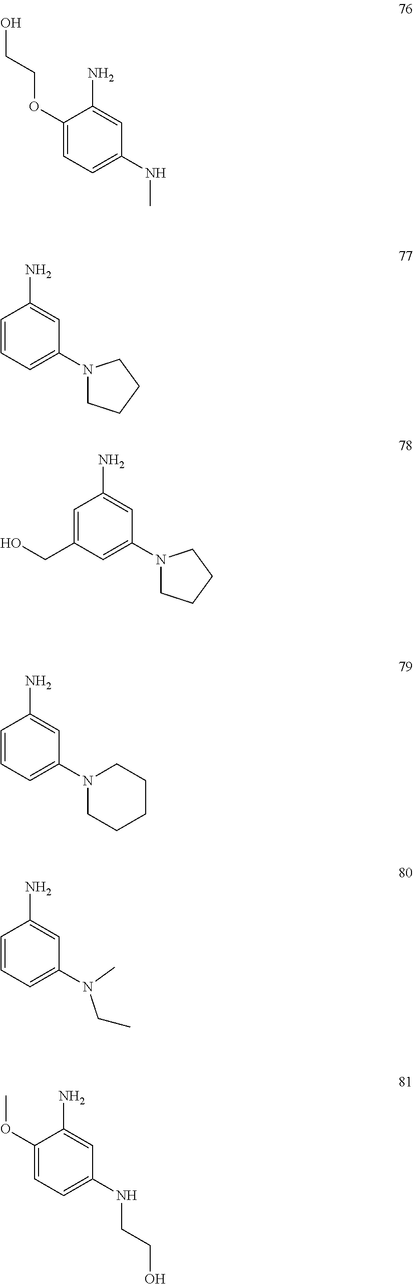 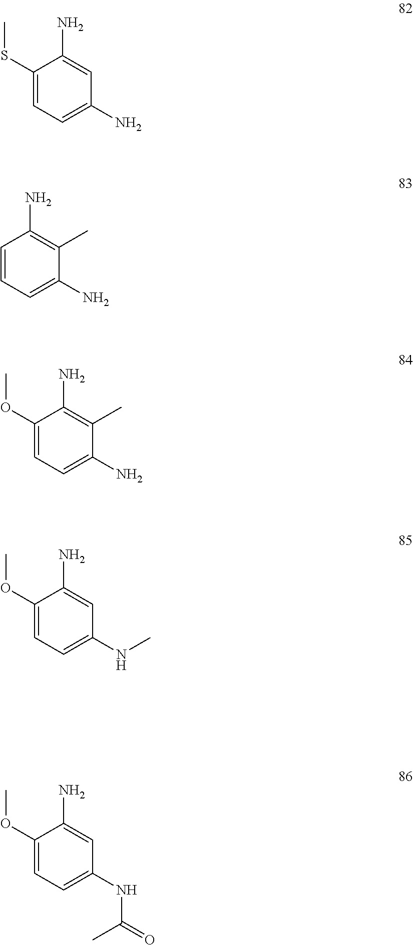
wherein An⁻ represents an anionic counterion.

22. The method according to claim 21, wherein the meta-phenylenediamine couplers are chosen from those of formulae 3, 9, 15, 51, and 81, and also the organic or inorganic acid salts thereof, the optical or geometrical isomers thereof, the tautomers or solvates thereof, or the hydrates thereof.

23. The method according to claim 21, wherein An is chosen from halide anionic counterions or Cl⁻.

24. The method according to claim 17, wherein the at least one dyeing agent is chosen from poly(2-(5-amino-2-methoxyphenylamino)ethanol), poly(2-(2,4-diaminophenoxy)ethanol), poly(2,4-dimethylbenzene-1,3-diamine), or poly(4-methylbenzene-1,3-diamine).

25. The method according to claim 17, wherein the at least one ingredient ii) is chosen from those of formulae (V) and (VI), and also the organic or inorganic acid salts thereof, the optical or geometrical isomers thereof, the tautomers or solvates, or the hydrates thereof: 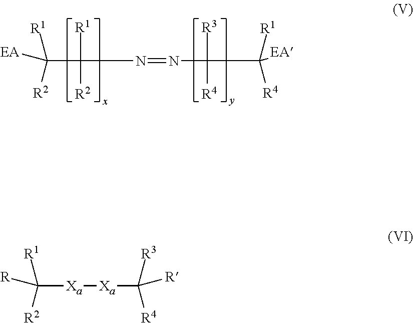
wherein in formulae (V) and (VI):
R¹, R², R³ and R⁴, which are identical or different, are chosen from a hydrogen atom or a linear or branched (C₁-C₈)alkyl group which is optionally substituted, a linear or branched (C₁-C₈)alkoxy group, an optionally substituted aryl group, or an optionally substituted phenyl;
or R₁ and R₂ and/or R₃ and R₄ form, together with the carbon atoms which carry them, an optionally substituted 3- to 7-membered (hetero)cycloalkyl, a (C₃-C₆)cycloalkyl, or cyclohexyl;
x and y, which are identical or different, represent an integer ranging from 0 to 6 inclusive;
EA and EA′, which are identical or different, represent an electron-withdrawing group;
R and R′, which are identical or different, represent i) an EA or EA′ radical as defined above, or a group chosen from ii) optionally substituted and linear or branched (C₁-C₈)alkyl groups, iii) optionally substituted aryl groups, or iv) optionally substituted aryl(C₁-C₈)alkyl groups,
or else R with R¹ and/or R′ with R³ form, together with the carbon atoms which carry them, a C(X¹) group, with R² and R⁴ being as defined above, or R² and R⁴, which are identical or different, represent an R⁵—(X²)_{w}— group in which w has the value 0 or 1; R⁵ is chosen from a hydrogen atom, a linear or branched (C₁-C₈)alkyl group, an optionally substituted aryl or heteroaryl group, optionally substituted phenyl, a cycloalkyl or heterocycloalkyl group which is optionally substituted, a cycloalkyl or heterocycloalkyl group which is optionally substituted by a (C₁-C₆)alkyl group, or cyclohexyl optionally substituted by a (C₁-C₆)alkyl group; and X² is as defined below;
Xₐ, which are identical or different, is chosen from a heteroatom chosen from oxygen and sulfur, a —C(O)—O— or —O—C(O)— group, a —O—C(O)—O— group, or —O—C(O)—O— group;
X¹ and X², which are identical or different, represent a heteroatom chosen from oxygen, sulfur, or amino N(R″) with R″ being a hydrogen atom or a linear or branched (C₁-C₆)alkyl group.

26. The method according to claim 17, wherein the at least one ingredient ii) is chosen from:
inorganic or organic persulfates, or ammonium persulfates;
organic peroxides, di(tert-butyl) peroxide, benzoyl peroxide (BPO), dicumyl peroxide, or methyl ethyl ketone peroxide (MEKP);
azo compounds, such as 2,2′-azobisisobutyronitrile (AIBN) or 1,1′-azobis (cyclohexanecarbonitrile) (ABCN); and
the products of reaction:
between hydrogen peroxide and ascorbic acid;
between hydrogen peroxide and metal salts,
between hydrogen peroxide and Fe²⁺ metal salts;
between organic peroxides, ammonium persulfate, or metabisulfites;
alkali metal or alkaline earth metal dichromates, or sodium dichromate; or
copper halides, Cu(II) copper halides, or CuCl₂.

27. The method according to claim 17, wherein the at least one ingredient ii) is chosen from those of formula (VII): 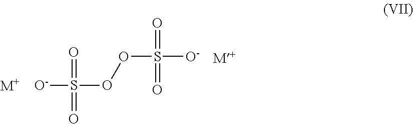
wherein in formula (VII):
M⁺ represents an inorganic or organic cationic counterion chosen from cations of alkali metals, sodium, potassium, or cations of alkaline earth metals, and ammonium R^{a}R^{b}R^{c}R^{d}N⁺, with R^{a}, R^{b}, R^{c} and R^{d}, which are identical or different, chosen from a hydrogen atom, a (C₁-C₆)alkyl group, or NH₄⁺.

28. The method according to claim 17, further comprising:
leaving the at least one dyeing agent to stand on the keratinous substance for a period of time ranging from about 5 minutes to about 1 hour inclusive;
optionally shampooing the keratinous substance;
optionally rinsing the keratinous substance; and
optionally drying the keratinous substance.

29. A method for producing at least one dyeing agent, the method comprising:
dissolving at least two meta-phenylenediamine couplers in a liquid solution to form a meta-phenylenediamine solution;
adding at least one alkaline agent to the meta-phenylenediamine solution to form a meta-phenylenediamine solution having a basic pH;
adding at least one ingredient chosen from radical initiators and chemical oxidizing agents to the meta-phenylenediamine solution to form a reaction solution; and
optionally, filtering off any precipitate that is formed, washing the precipitate, and/or drying the precipitate.

30. The method according to claim 29, further comprising at least one condition chosen from:
the liquid solution is an aqueous/alcoholic solution;
the at least one alkaline agent is aqueous ammonia;
the meta-phenylenediamine solution has a pH ranging from about 8 to about 10;
the method is carried out at ambient temperature;
the reaction solution is stirred for a period of time ranging from about 30 minutes to about 2 days;
the precipitate is washed with water; and
the precipitate is dried under vacuum.

31. The method according to claim 29, wherein the weight ratio between the total amount of meta-phenylenediamine couplers and the total amount of one ingredient chosen from radical initiators and chemical oxidizing agents ranges from about 0.5 to about 1.5.

32. The method according to claim 29, wherein the weight ratio between the total amount of meta-phenylenediamine couplers and the total amount of one ingredient chosen from radical initiators and chemical oxidizing agents is about 1.

33. A dyeing agent obtained from the polycondensation between i) at least two meta-phenylenediamine couplers which react with one another in the presence of ii) at least one ingredient chosen from radical initiators and chemical oxidizing agents.

34. The dyeing agent according to claim 33, wherein the dyeing agent is chosen from poly(2-(5-amino-2-methoxyphenylamino)ethanol), poly(2-(2,4-diaminophenoxy)ethanol), poly(2,4-dimethylbenzene-1,3-diamine), poly(4-methylbenzene-1,3-diamine), those of formula (Ia) or the salts of inorganic or organic acids or bases thereof, the optical isomers or geometrical isomers thereof, the tautomers and/or solvates thereof, or the hydrates thereof: 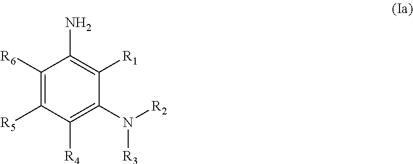
wherein in formula (Ia):
R₂ and R₃, which are identical or different, are chosen from:
i) a hydrogen atom;
ii) a (C₁-C₆)alkyl group optionally substituted by at least one group chosen from: a) hydroxyl, b) urea —N(H)—C(O)—NH₂, c) guanidino —N(H)—C(NH)—NH₂, d) (di)(C₁-C₄)(alkyl)amino, or e) (C₁-C₄)alkylthio;
iii) a (C₁-C₄)alkylcarbonyl group, or
iv) an aminocarbonyle H₂N—C(O)— group;
or R₂ and R₃ form, together with the nitrogen atom which carries them, a saturated heterocycle;
R₁, R₄, R₅, and R₆, which are identical or different, are chosen from
i) a hydrogen or halogen atom,
ii) a (C₁-C₆)alkyl radical optionally substituted by at least one atom or group chosen from: a) halogen, b) hydroxyl, c) (di)(C₁-C₆)(alkyl)amino, or d) (C₁-C₆)alkylthio,
iii) (C₁-C₆)alkoxy groups, optionally substituted by at least one halogen atom or at least one group chosen from hydroxyl, (di)(C₁-C₄)(alkyl)amino, carboxyl, (C₁-C₆)alkylcarbonyl, R—C(O)—N(R′)—, R—S(O)₂—N(R′)—, RR′N—C(O)—, cationic heterocycle or cationic heteroaryl, N—(C₁-C₄)alkylpyrrolidinium, N—(C₁-C₄)alkylpiperidinium, N,N′-di(C₁-C₄)alkylpiperazinium, N—(C₁-C₄)alkylmorpholinium or N—(C₁-C₄)alkylimidazolium, or tri(C₁-C₄)alkylammonium groups, with R and R′, which are identical or different, chosen from a hydrogen atom or a (C₁-C₄)alkyl group, with the proviso that the alkyl group of the alkoxy is optionally interrupted by at least one oxygen atom;
iv) (C₁-C₆)alkylthio groups, optionally substituted by at least one group chosen from hydroxyl, (di)(C₁-C₄)(alkyl)amino or R—C(O)—N(R′)—, with R and R′ as defined above;
v) R—C(O)—N(R′)—, with R and R′ as defined above;
vi) R—O—C(O)— with R as defined above;
vii) carboxyl;
viii) SO₃H; or
ix) aminocarbonyl;
with the proviso that, when the compound of formula (Ia) comprises at least one cationic substituent, the compound comprises as many anionic counterions as cationic charges in order to achieve the electrical neutrality of the compound of formula (Ia).

35. A composition comprising the at least one dyeing agent according to claim 33.

36. The composition according to claim 35, wherein the composition does not comprise an oxidation base and/or a chemical oxidizing agent.
